(19) United States
(12) Patent Application Publication
(10) Pub. No.: US 2022/0023227 A1
(43) Pub. Date: Jan. 27, 2022

(54) SYSTEMS AND METHODS FOR FORMING NANOCAPSULES BY PULSED ELECTROSPRAYING

(71) Applicant: Georgia Southern University Research and Service Foundation, Inc., Statesboro, GA (US)

(72) Inventors: Mujibur Rahman Khan, Martinez, GA (US); Md Mahmudul Hasan, Urbana, IL (US); Ali S. Arbab, Martinez, GA (US); Mohammad Harun Rashid, Augusta, GA (US)

(21) Appl. No.: 17/328,708

(22) Filed: May 24, 2021

Related U.S. Application Data
(60) Provisional application No. 63/056,228, filed on Jul. 24, 2020.

(51) Int. Cl. A61K 9/51; B01J 13/04; A61K 31/337; B05B 5/025

(52) U.S. Cl. CPC A61K 9/5192; B01J 13/04; B82Y 5/00; B05B 5/0255; A61K 31/337

(57) ABSTRACT

Disclosed herein are nano-encapsulated compositions and systems and methods of preparing the same. The compositions may be obtained through co-axial electrospraying with pulsating voltage. The nano-encapsulated composition exhibit improved pharmacokinetic properties. Various embodiments include systems and methods that apply a constant voltage and/or a pulsating voltage at a frequency to a plate having co-axial outlets through which a core solution and capsule solution flow. In some implementations, the pulsating voltage fluctuates between a minimum pulsating voltage and a maximum pulsating voltage, wherein the minimum pulsating voltage is 0 KV or greater and the maximum pulsating voltage is greater than 0 KV and the minimum pulsating voltage. The constant voltage is greater than 0 KV, and a total maximum applied voltage to the plate is the sum of the maximum pulsating voltage and the constant voltage.

### CROSS-REFERENCE TO RELATED APPLICATION

This application claims the benefit of U.S. Provisional Application 63/056,228, filed Jul. 24, 2020, the contents of which are hereby incorporated in its entirety.

### STATEMENT REGARDING FEDERALLY SPONSORED RESEARCH OR DEVELOPMENT

This invention was made with government support under Grant No. DMR 1337545 awarded by the National Science Foundation's Major Research Instrumentation Program (NSF MRI). The government has certain rights in this invention.

### BACKGROUND

Electrospraying is a technique for aerosolizing a liquid. Liquid is supplied through a capillary and a high voltage is applied to the tip of the capillary. Spaced apart from the tip is a collector plate that is grounded or to which low voltage is applied. The high potential at the tip results in the formation of a Taylor cone, whereby a liquid jet is emitted through the apex of the cone. The jet rapidly forms into droplets as a result of Coulombic repulsion in the fluid.

Nanomaterials such as nanoparticles have been the subject of substantial biomedical research over the last two decades, especially as carriers for drug delivery systems, for instance either as implantable, transdermal or fast dissolving systems. Selective delivery of nanoparticles directly to cancerous cells or tumors could mitigate many of the harmful side effects of chemotherapy. Furthermore, it has been reported that certain nanoparticles may selectively cross biological barriers, for instance the blood brain barrier. Nanoparticles may also be able to selectively access tumor cells on the premise that leaky vasculature present in tumor tissue may admit appropriately sized particles. This observation has been designated the Enhanced Permeability and Retention effect (“EPR effect”), which has been demonstrated with thermal ablation of gold nanoparticles.

Nanoparticles have conventionally been prepared using precipitation or emulsion polymerization techniques. These methods highly depend on the polymer and drug characteristics such as molecular weight, biodegradability, and hydrophobicity. These chemical methods are often characterized by poor encapsulation efficiency, and it can be hard to control drug loading, resulting in inconsistent efficacy and pharmacokinetic properties. Also, these methods are extremely difficult to conduct on large scales, which presents a high barrier for clinical use. The outcome of any given emulsion polymerization is affected by multiple characteristics such as copolymer composition, chemical composition distribution, molar mass distribution, polymer architecture, particle morphology, particle size distribution and surface composition. It can be difficult to precisely control the thickness of the capsule shell, which is often governed by thermodynamic factors inherent to the individual system. Moreover, not all drug substances may be encapsulated due to incompatibility with the solvent system, a problem which is compounded if multiple drugs, having different solubilities, are intended to be encapsulated together. Finally, the encapsulated drug is invariably accompanied with solvent or other vehicle (for instance an oil or aqueous phase) from the encapsulation process. As chemical reactions (including degradation reactions) are generally faster in the presence of a solvent, drug stability is a concern for nanocapsules prepared by emulsion polymerization.

There remains a need for nanomaterials like nanoparticles with improved properties, for instance improved stability, as drug carriers with improved release characteristics and/or increased potency. There is a need for improved methods of preparing nanoparticles with high degrees of uniformity, as well as to control the thickness of the capsule shell.

### SUMMARY OF THE INVENTION

Various implementations include a method of forming nanocapsules by pulsed, coaxial electrospraying. The method includes providing at least one core solution comprising at least one solvent and at least one active agent; providing at least one capsule solution including at least one solvent and at least one polymer; coaxially electrospraying the core solution and capsule solution through a plate that defines a core channel and a capsule channel, the core channel having a core outlet and the capsule channel having a capsule outlet, wherein the capsule outlet is coaxial with the core outlet and is disposed radially outwardly from the core outlet; and collecting the formed nanocapsules on a grounded collection plate spaced apart from the plate, wherein the coaxial electrospraying includes: applying a pulsating voltage at a frequency to the plate, the pulsating voltage fluctuating between a minimum pulsating voltage and a maximum pulsating voltage, wherein the minimum pulsating voltage is 0 KV or greater and the maximum pulsating voltage is greater than the minimum pulsating voltage; and applying a constant voltage to the plate, the constant voltage being greater than 0 KV, the maximum pulsating voltage being greater than 0 KV, and a total maximum applied voltage to the plate being the sum of the maximum pulsating voltage and the constant voltage.

In some implementations, the at least one core solution includes: a first core solution including at least one solvent and at least one first active agent; and a second core solution including at least one solvent and at least one second active agent, wherein the method further includes combining the first core solution and second core solution in a core solution manifold prior to coaxially electrospraying the core solutions through the plate.

In some implementations, the first core solution is immiscible in the second core solution.

In some implementations, the first core solution and the second core solution are pumped to the core solution manifold by at least one core solution pump.

In some implementations, a flow rate of the first core solution into the core solution manifold is greater than a flow rate of the second core solution into the core solution manifold.

In some implementations, the capsule solution includes at least: a first capsule solution including at least one solvent and at least one polymer; and a second capsule solution including at least one solvent and at least one additional capsule agent, wherein the method further includes combining the first capsule solution and second capsule solution in a capsule solution manifold prior to coaxially electrospraying the capsule solutions through the plate.

In some implementations, the first capsule solution is immiscible in the second capsule solution.

In some implementations, the first capsule solution and the second capsule solution are pumped to the capsule solution manifold by at least one capsule solution pump.

In some implementations, the minimum pulsating voltage is 0 KV and the maximum pulsating voltage is less than or equal to 50 KV.

In some implementations, the maximum pulsating voltage is less than or equal to 40 KV. In some implementations, the method further includes sonicating one or more of the core solution, capsule solution, or precursor solutions thereof, prior to co-axial electrospraying.

In some implementations, the active agent of the core solution includes at least one chemotherapeutic agent. In some implementations, the chemotherapeutic agent includes paclitaxel.

In some implementations, the active agent of the core solution includes a tumor microenvironment (TME) altering agent.

In some implementations, the TME altering agent includes GW2580.

In some implementations, the at least one polymer of the capsule solution includes one or more biocompatible, biodegradable polymers.

In some implementations, the biocompatible, biodegradable polymer includes one or more polyester, mixed polyester, polyanhydride, mixed polyanhydride, poly(ester)anhydride, polysaccharide, polyphosphazene or polyphosphoester.

In some implementations, the biocompatible, biodegradable polymer includes PLGA, polycaprolactone, polylactide, polyglycolide, polyhydroxybutyric acid, poly(sebacic acid), poly[1,6-bis(p-carboxyphenoxy)hexane], polyvinylpyrrolidone, polyvinyl alcohol, poly(ethylene oxide), poly(propylene oxide), copolymers thereof, or a mixture thereof.

Various other implementations include a coaxial electrospraying system for forming nanocapsules. The electrospraying system includes at least one core solution pump in fluid communication with at least one core solution; at least one capsule solution pump in fluid communication with at least one capsule solution; a plate defining a core channel having a core outlet and a capsule channel having a capsule outlet, the core outlet and capsule outlet being coaxial and the capsule outlet being disposed radially outward of the core outlet, the core outlet being in fluid communication with the at least one core solution pump, and the capsule outlet being in fluid communication with the at least one capsule solution pump; a pulsating voltage source electrically coupled to the plate, the pulsating voltage source configured to apply a pulsed voltage that fluctuates between a minimum pulsating voltage and a maximum pulsating voltage, wherein the minimum pulsating voltage is 0 KV or greater, and the maximum pulsating voltage is greater than 0 KV and the minimum pulsating voltage; a constant voltage source electrically coupled to the plate, the constant voltage source applying a constant voltage to the plate, the constant voltage being greater than 0 KV, and wherein a total maximum applied voltage to the plate is the sum of the maximum pulsating voltage and the constant voltage; and a collector that faces an outlet side of the plate, is spaced apart therefrom, and is grounded relative to the pulsating voltage source and the constant voltage source.

In some implementations, the pulsating voltage source is electrically coupled to a transistor switch, wherein the transistor switch pulses the pulsating voltage between the minimum pulsating voltage and the maximum pulsating voltage at the frequency.

In some implementations, the maximum pulsating voltage is less than or equal to 50 KV.

In some implementations, the system further includes a core solution manifold disposed between the at least one core solution pump and the plate, wherein the core solution manifold has two or more inlet openings and a single exit opening, each inlet opening is configured for receiving at least one core solution, and the single exit opening is in fluid communication with the core outlet.

In some implementations, the system further includes a capsule solution manifold disposed between the at least one capsule solution pump and the plate, the capsule solution manifold having two or more inlet openings and a single exit opening, each inlet opening of the capsule solution manifold is configured for receiving at least one capsule solution, and the single exit opening is in fluid communication with the capsule outlet.

In some implementations, the system further includes a controller for controlling a flow rate of the at least one core solution pump and a flow rate of the at least one capsule solution pump, wherein a flow rate from the single exit opening of the capsule solution manifold is greater than a flow rate from the single exit opening of the core solution manifold.

In some implementations, the system further includes a controller for controlling a flow rate of the at least one core solution pump and a flow rate of the at least one capsule solution pumps.

Various other implementations include a nanocapsule prepared by any of the aforementioned methods or systems.

Various other implementations include a method of forming nanocapsules by coaxial electrospraying. The method includes providing two or more core solutions, each core solution including at least one solvent and at least one active agent; providing at least one capsule solution including at least one solvent and at least one polymer; combining the two or more core solutions in a core solution manifold, coaxially electrospraying the capsule solution and the combined core solutions through a plate that defines a core channel and a capsule channel, the core channel having a core outlet and the capsule channel having a capsule outlet, wherein the capsule outlet is coaxial with the core outlet and is disposed radially outwardly from the core outlet; and collecting the formed nanocapsules on a grounded collection plate spaced apart from the plate, wherein the coaxial electrospraying includes applying a constant voltage to the plate, the constant voltage being greater than 0 KV.

In some implementations, the two or more core solutions include a first core solution and a second core solution, and the first core solution is immiscible in the second core solution.

In some implementations, the two or more core solutions are pumped to the core solution manifold by at least one core solution pump.

In some implementations, the capsule solution includes: a first capsule solution including at least one solvent and at least one polymer; and a second capsule solution including at least one solvent and at least one additional capsule agent, and the method further includes combining the first capsule solution and the second capsule solution in a capsule solution manifold prior to coaxially electrospraying the combined capsule solution.

In some implementations, the first capsule solution is immiscible in the second capsule solution.

In some implementations, the first capsule solution and the second capsule solution are pumped to the capsule solution manifold by at least one capsule solution pump.

In some implementations, the method further includes sonicating one or more of the core solutions, capsule solution, or precursor solutions thereof, prior to co-axial electrospraying.

In some implementations, the active agent of at least one of the at least two core solutions includes at least one chemotherapeutic agent. In some implementations, the chemotherapeutic agent includes paclitaxel.

In some implementations, the active agent of at least one of the at least two core solutions include a tumor microenvironment (TME) altering agent. In some implementations, the TME altering agent includes GW2580.

In some implementations, the at least one polymer of the capsule solution includes one or more biocompatible, biodegradable polymers.

In some implementations, the at least one polymer of the capsule solution includes a polymer having a molecular weight of less than 14,000 grams per mole.

In some implementations, the biocompatible, biodegradable polymer includes one or more polyester, mixed polyester, polyanhydride, mixed polyanhydride, poly(ester)anhydride, polysaccharide, polyphosphazene or polyphosphoester.

In some implementations, the biocompatible, biodegradable polymer includes PLGA, polycaprolactone, polylactide, polyglycolide, polyhydroxybutyric acid, poly(sebacic acid), poly[1,6-bis(p-carboxyphenoxy)hexane], polyvinylpyrrolidone, polyvinyl alcohol, poly(ethylene oxide), poly(propylene oxide), copolymers thereof, or a mixture thereof.

In some implementations, a flow rate of the first core solution into the core solution manifold is greater than a flow rate of the second core solution into the core solution manifold.

Various other implementations include a coaxial electrospraying system for forming nanocapsules. The electrospraying system includes: at least one core solution pump in fluid communication with two or more core solutions; a core solution manifold in fluid communication with the at least one core solution pump, the core solution manifold having two or more inlet openings and a single exit opening, each inlet opening being configured for receiving at least one of the two or more core solutions; at least one capsule solution pump in fluid communication with at least one capsule solution; a plate defining a core channel having a core outlet and a capsule channel having capsule outlet, the core outlet and capsule outlet being coaxial and the capsule outlet being radially outward of the core outlet, the core outlet being in fluid communication with the single exit opening of the core solution manifold and the at least one core solution pump, and the capsule outlet being in fluid communication with the at least one capsule solution pump; a constant voltage source electrically coupled to the plate, the constant voltage source applying a constant voltage to the plate, the constant voltage being greater than 0 KV; and a collector that faces an outlet side of the plate, is spaced apart therefrom, and is grounded relative to the pulsating voltage source and the constant voltage source.

In some implementations, the system further includes a capsule solution manifold in fluid communication with the at least one capsule solution pump for receiving two or more capsule solutions, the capsule solution manifold having two or more inlet openings and a single exit opening, each inlet opening being configured for receiving at least one of the two or more capsule solutions, and the single exit opening of the capsule solution manifold being in fluid communication with the capsule outlet.

In some implementations, the system further includes a controller for controlling a flow rate of the at least one core solution pump and a flow rate of the at least one capsule solution pump.

In some implementations, a flow rate from the single exit opening of the capsule solution manifold is greater than a flow rate from the single exit opening of the core solution manifold.

Various other implementations include a nanocapsule, prepared by any of the aforementioned methods or systems.

Various other implementations include a method of forming nanocapsules by pulsed, coaxial electrospraying. The method includes: providing at least one core solution including at least one solvent and at least one active agent; providing at least one capsule solution including at least one solvent and at least one polymer; coaxially electrospraying the core solution and capsule solution through a plate that defines a core channel and a capsule channel, the core channel having a core outlet and the capsule channel having a capsule outlet, wherein the capsule outlet is coaxial with the core outlet and is disposed radially outwardly from the core outlet; and collecting the formed nanocapsules on a grounded collection plate spaced apart from the plate, wherein the coaxial electrospraying includes applying a pulsating voltage at a frequency to the plate, the pulsating voltage fluctuating between a minimum pulsating voltage and a maximum pulsating voltage, wherein the minimum pulsating voltage is greater than 0 KV and the maximum pulsating voltage is greater than the minimum pulsating voltage.

In some implementations, the at least one core solution includes: a first core solution including at least one solvent and at least one first active agent; and a second core solution including at least one solvent and at least one second active agent, wherein the method further includes combining the first core solution and second core solution in a core solution manifold prior to coaxially electro spraying the core solutions.

In some implementations, the first core solution is immiscible in the second core solution. In some implementations, the first core solution and the second core solution are pumped to the core solution manifold by at least one core pump.

In some implementations, a flow rate of the first core solution into the core solution manifold is greater than a flow rate of the second core solution into the core solution manifold.

In some implementations, the at least one capsule solution includes: a first capsule solution including at least one solvent and at least one polymer; and a second capsule solution including at least one solvent and at least one additional capsule agent, wherein the method further includes combining the first capsule solution and second capsule solution in a capsule solution manifold prior to coaxially electrospraying the capsule solutions.

In some implementations, the first capsule solution is immiscible in the second capsule solution.

In some implementations, the first capsule solution and the second capsule solution are pumped to the capsule solution manifold by at least one capsule pump.

In some implementations, the minimum pulsating voltage is greater than 0 KV and the maximum pulsating voltage is less than or equal to 50 KV.

In some implementations, the maximum pulsating voltage is greater than 0 KV and less than or equal to 40 kV.

In some implementations, the method further includes sonicating one or more of the core solution, capsule solution, or precursor solutions thereof, prior to co-axial electrospraying.

In some implementations, the active agent of the at least one core solution includes at least one chemotherapeutic agent.

In some implementations, the chemotherapeutic agent includes paclitaxel.

In some implementations, the active agent of the at least one core solution includes a tumor microenvironment (TME) altering agent.

In some implementations, the TME altering agent includes GW2580.

In some implementations, the at least one polymer of the at least one capsule solution includes one or more biocompatible, biodegradable polymers.

In some implementations, the biocompatible, biodegradable polymer includes one or more polyester, mixed polyester, polyanhydride, mixed polyanhydride, poly(ester)anhydride, polysaccharide, polyphosphazene or polyphosphoester.

In some implementations, the biocompatible, biodegradable polymer includes PLGA, polycaprolactone, polylactide, polyglycolide, polyhydroxybutyric acid, poly(sebacic acid), poly[1,6-bis(p-carboxyphenoxy)hexane], polyvinylpyrrolidone, polyvinyl alcohol, poly(ethylene oxide), poly(propylene oxide), copolymers thereof, or a mixture thereof.

Various other implementations include a coaxial electrospraying system for forming nanocapsules. The electrospraying system includes: at least one core solution pump in fluid communication with at least one core solution; at least one capsule solution pump in fluid communication with at least one capsule solution; a plate defining a core channel having a core outlet and a capsule channel having capsule outlet, the core outlet and capsule outlet being coaxial and the capsule outlet being disposed radially outward of the core outlet, the core outlet being in fluid communication with the at least one core solution pump, and the capsule outlet being in fluid communication with the at least one capsule solution pump; a pulsating voltage source electrically coupled to the plate, the pulsating voltage source configured to apply a pulsed voltage that fluctuates between a minimum pulsating voltage and a maximum pulsating voltage, wherein the minimum pulsating voltage is greater than 0 KV, and the maximum pulsating voltage is greater than 0 KV and the minimum pulsating voltage; and a collector that faces an outlet side of the plate, is spaced apart therefrom, and is grounded relative to the pulsating voltage source and the constant voltage source.

In some implementations, the pulsating voltage source further includes a transistor switch, wherein the transistor switch pulses the pulsating voltage between the minimum pulsating voltage and the maximum pulsating voltage at the frequency.

In some implementations, the maximum pulsating voltage is greater than 0 KV and less than or equal to 50 kV.

In some implementations, the system further includes a core solution manifold disposed between the at least one core solution pump and the plate, the core solution manifold having two or more inlet openings and a single exit opening, each inlet opening is configured for receiving at least one core solution, and the single exit opening is in fluid communication with the core outlet.

In some implementations, the system further includes a capsule solution manifold disposed between the at least one capsule solution pump and the plate, the capsule solution manifold having two or more inlet openings and a single exit opening, each inlet opening being configured for receiving at least one capsule solution, and the single exit opening, being in fluid communication with the capsule outlet.

In some implementations, the system further includes a controller for controlling a flow rate of the at least one core solution pump and a flow rate of the at least one capsule solution pump, wherein a flow rate from the single exit opening of the capsule solution manifold is greater than a flow rate from the single exit opening of the core solution manifold.

In some implementations, the system further includes a controller for controlling a flow rate of the at least one core solution pump and a flow rate of the at least one capsule solution pump.

Various other implementations include a nanocapsule prepared by any of the aforementioned methods of systems.

In any of the above implementations, the plate comprises a concentric nozzle that defines the core channel and the capsule channel, and the concentric nozzle is coupled to and in electrical communication with the plate.

Other systems, methods, features and/or advantages will be or may become apparent to one with skill in the art upon examination of the following drawings and detailed description. It is intended that all such additional systems, methods, features and/or advantages be included within this description and be protected by the accompanying claims.

### BRIEF DESCRIPTION OF THE FIGURES

FIG. 1: Intratumoral administration of encapsulated paclitaxel with GW2558 increased the survival of mice implanted with mammary tumor. Highly aggressive 4T1 tumor cells were implanted into 4th mammary fat pad of BALB/c mice and treatment was started one week post-implantation and total 3 doses (one dose/week) were injected. (a) Three doses injection of encapsulated paclitaxel plus GW2558 increased the survival mice while all naked paclitaxel injected mice died within 62 days. (b) However, there were no significant difference in tumor size up to 42 days between groups. Results are presented as mean±SD (3-5 mice in each group).

FIG. 2: Intratumoral administration of encapsulated paclitaxel with GW2558 did not alter the metastatic potential of 4T1 tumor cells. Mice were sacrificed 3 days after the last dose injection to check the metastatic colonization of luciferase expressing 4T1 tumor cells. (a-b) IP-injection of naked paclitaxel decrease the tumor weight. (c) Although encapsulated pac (PCL-Pac+GW) showed a tendency towards increased weight, no significant difference were detected for spleen weight between groups. (d-e) While only micro metastases were observed in the lungs of naked pac-treated animals, two of animals in encapsulated pac-treated group showed macro metastasis in their lungs. Yet, signal intensity of luciferase was not significantly different between groups. Results are presented as mean±SD (3 mice in each group).

FIG. 3 depicts SEM images particle transitions.

FIG. 4 depicts coaxial electrospraying sample.

FIG. 5 depicts a drug release over time of paclitaxel from nanocapsules having a PCL shell and a paclitaxel core.

FIG. 6: depicts in vitro efficacy of paclitaxel loaded PCL nanoparticle produced in coaxial electrospraying.

FIGS. 7A and 7B depict in-vitro drug efficacy test for paclitaxel and GW-2580.

FIG. 8 depicts TEM images for coaxial electro sprayed multidrug sample nanocapsules.

FIG. 9 depicts UV-Vis absorbance spectrum for different days (paclitaxel-227 nm; GW2580-227 nm, 280 nm).

FIG. 10A depicts a plan side view of a schematic diagram of a co-axial electrospraying system for forming nanocapsules, according to one implementation.

FIG. 10B depicts enlarged side views and associated insets of the co-axial electrospraying system for forming nanocapsules of FIG. 10A.

FIG. 10C depicts a cross sectional view of the plate shown in FIG. 10A.

FIG. 11A depicts a schematic diagram of the electronics associated with voltage control in the system of FIG. 10A.

FIG. 11B illustrates a transistor switch and a voltage versus time diagram for pulsed electro spraying, according to one implementation.

FIG. 11C depicts a schematic diagram of the dual power systems providing respective pulsating voltages and constant voltages to the electronics of FIG. 11A.

FIG. 12 illustrates a percentage of drug released over a two-week period, according to one example.

FIG. 13 illustrates results of a cell viability test showing the efficacy of the drug nanocrystals produced by pulsed electrospraying using the system shown in FIGS. 10A-11C.

FIG. 14 illustrates TEM images of the nanocapsules produced using the system shown in FIGS. 10A-11C. The nanocapsules have a core of Paclitaxel and GW2580 and a shell of PCL. TEM images are shown before filtering.

FIG. 15 illustrates in vivo tumor growth (luciferase+4T1 breast cancer) and animal survival using the PCL-Paclitaxel-GW2580 nanocapsules produced by the system in FIGS. 10A-11C.

FIG. 16 illustrates electrosprayed PCL-PEG-NH₂ nanoparticles tagged with fluorescent dye and in vivo bio distribution.

FIG. 17 illustrates UV-vis detection of the peptides in the peptides-PCL nanoparticles, demonstrating successful incorporation of peptides onto the electrosprayed nanoparticles.

FIG. 18 depicts individual peptide/PCL nanocapsules (100 nm) and agglomerated nanocapsules (500 nm-800 nm).

### DETAILED DESCRIPTION

Before the present methods and systems are disclosed and described, it is to be understood that the methods and systems are not limited to specific synthetic methods, specific components, or to particular compositions. It is also to be understood that the terminology used herein is for the purpose of describing particular embodiments only and is not intended to be limiting.

As used in the specification and the appended claims, the singular forms “a,” “an” and “the” include plural referents unless the context clearly dictates otherwise. Ranges may be expressed herein as from “about” one particular value, and/or to “about” another particular value. When such a range is expressed, another embodiment includes from the one particular value and/or to the other particular value. Similarly, when values are expressed as approximations, by use of the antecedent “about,” it will be understood that the particular value forms another embodiment. It will be further understood that the endpoints of each of the ranges are significant both in relation to the other endpoint, and independently of the other endpoint. As used herein, when a value is given as between a first and second number, the range includes the first and second numbers.

“Optional” or “optionally” means that the subsequently described event or circumstance may or may not occur, and that the description includes instances where said event or circumstance occurs and instances where it does not.

Throughout the description and claims of this specification, the word “comprise” and variations of the word, such as “comprising” and “comprises,” means “including but not limited to,” and is not intended to exclude, for example, other additives, components, integers or steps. “Exemplary” means “an example of” and is not intended to convey an indication of a preferred or ideal embodiment. “Such as” is not used in a restrictive sense, but for explanatory purposes.

Disclosed are components that can be used to perform the disclosed methods and systems. These and other components are disclosed herein, and it is understood that when combinations, subsets, interactions, groups, etc. of these components are disclosed that while specific reference of each various individual and collective combinations and permutation of these may not be explicitly disclosed, each is specifically contemplated and described herein, for all methods and systems. This applies to all aspects of this application including, but not limited to, steps in disclosed methods. Thus, if there are a variety of additional steps that can be performed it is understood that each of these additional steps can be performed with any specific embodiment or combination of embodiments of the disclosed methods.

Disclosed herein are various embodiments of nanocapsules that include a core encapsulated by one or more polymers. The core can include at least one active agent, e.g., a therapeutic agent or a diagnostic agent, e.g., radionuclides, fluorophores, dyes, and the like. The therapeutic agent can include cytotoxic drugs, antibiotics, immunosuppres sant, chemotherapy adjuvants, as well as biopolymers such as proteins and antibodies, vaccines, and nucleic acids. The polymer can be a biodegradable, biocompatible polymer, and one or more active agents can be dispersed in the polymer shell (also referred to herein as “capsule” or “capsule shell”). In some embodiments, targeting moieties and/or other active agents (as defined herein) can be appended to the surface of the polymer shell. The targeting moieties and/or other active agents may be appended to the surface via covalent bonds, e.g., using functionalized polymers and bioconjugation chemistries. In other embodiments, the targeting moieties and/or other active agents may be non-covalently attached to the surface of the nanocapsules. In some embodiments, a nanocapsule is formed by pulsed co-axial electrospraying, and the nanocapsule includes a nanocrystalline drug encapsulated by a biodegradable, biocompatible polymer.

The nanocapsules can be characterized by at least one diameter (d), which refers to the average particle diameter of the drug and polymer together. The nanocapsules described herein can have a diameter (d) no greater than about 1,000 nm, no greater than about 900 nm, no greater than about 800 nm, no greater than about 700 nm, no greater than about 600 nm, no greater than about 500 nm, no greater than about 400 nm, no greater than about 300 nm, no greater than about 200 nm, no greater than about 100 nm, no greater than about 75 nm, no greater than about 50 nm or no greater than about 25 nm. In certain embodiments, the nanocapsules described herein can have a diameter (d) from about 10-1,000 nm, about 10-900 nm, about 10-800 nm, about 10-700 nm, about 10-600 nm, about 10-500 nm, about 10-400 nm, about 10-300 nm, about 10-200 nm, about 10-100 nm, about 100-1,000 nm, about 100-750 nm, about 100-500 nm, 100-400 nm, 100-300 nm, about 100-250 nm, about 100-200 nm, about 25-200 nm, about 50-200 nm, or about 50-100 nm.

The nanocapsules described herein can be characterized by a high degree of uniformity. As used herein, the term “uniform” refers to a narrow distribution particle sizes. The distribution can be characterized by the standard deviation of the diameter (d). In some embodiments, the nanocapsules can be characterized by a standard deviation diameter (d) is no greater than 25%, 20%, 10%, 5%, 2.5% or 1% of the average diameter (d).

The nanocapsules can be characterized by a high encapsulation efficiency. As used herein, encapsulation efficiency can refer to the relative amount of polymer in the core, or the amount of pharmaceutically active agent in the capsule. In some embodiments, the core contains no more than 25%, 20%, 15%, 10%, 7.5%, 5.0%, 2.5%, 1% or 0.5% (w/w) of capsule polymer. In some embodiments, the capsule contains no more than 25%, 20%, 15%, 10%, 7.5%, 5.0%, 2.5%, 1% or 0.5% (w/w) of active agent.

Unlike nanocapsules prepared by emulsion polymerization, the nanocapsules disclosed herein do not include any aqueous or oil phase (or other solvent) in the core. In some embodiments, the core contains at least 50%, 60%, 70%, 80%, 90%, 95%, or 98% by weight active ingredient (either a single or multiple drug substances).

The combination of high encapsulation efficiency and uniform diameters provides controlled release of the pharmaceutically active agent from the core/capsule nanomaterial. Previous attempts to prepare nanocapsules for drug delivery often encountered burst release of the active agent upon exposure to a biological system. The nanocapsules can be characterized by the absence of “burst” release upon initial exposure to a biological system. The in vivo release profile can be estimated by measuring release in a system intended to mimic in vivo conditions. For instance, when the nanocapsules are immersed in 0.01 M PBS (phosphate buffered saline) (pH 7.4) at 37° C., no more than 0.1%, 0.5%, 1%, 2%, 3%, 4%, 5%, 6%, 7%, 8%, 9% or 10% of the pharmaceutically active agent is released within 24 hours. The rate of release can be controlled through proper selection of the biodegradable, biocompatible polymer as well as the relative thickness of the capsule material. In some embodiments, at least 50%, 60%, 65%, 70%, 75%, 80%, 85%, 90% or 95% of the pharmaceutically acceptable agent is released within a period of 7, 10, 14, 21, 28, 35, 42, 49, 56, 63, 70, 77, 84, 91 or 98 days.

In some embodiments, the pharmaceutically active agent (sometimes designated “drug”) can be a biological macromolecule, small molecule drug, or a mixture of two or more biological macromolecules and/or small molecule drugs. Unless explicitly specified to the contrary, the term “pharmaceutically active agent” embraces both single agents and mixtures of multiple agents. Typically, small molecule drugs are characterized by a molecular weight no greater than 1,000 Daltons. Exemplary classes of pharmaceutically active agents include cancer chemotherapeutics, immunosuppressants, antibiotics, analgesics, contraceptives, anesthetics, and the like. In certain embodiments, the pharmaceutically active agent can include one or more cancer chemotherapeutics, such as an alkylating agent, an antimetabolite, an anti-microtubule agent, a tyrosine kinase inhibitor, a topoisomerase inhibitor, a CSF-1R inhibitor, or a cytotoxic antibiotic.

In some instance, the chemotherapeutic agent can include alkylating agents, for instance, uracil mustard, chlormethine, cyclophosphamide, ifosfamide, melphalan, chlorambucil, pipobroman, triethylenemelamine, triethylenethiophosphoramine, temozolomide, thiotepa, busulfan, carmustine, lomustine, streptozocin, and dacarbazine, antimetabolites, for instance methotrexate, 5-fluorouracil, floxuridine, cytarabine, 6-mercaptopurine, 6-thioguanine, fludarabine phosphate, pentostatin, pemetrexed, raltitrexed, cladribine, clofarabine, mercaptopurine, capecitabine, nelarabine, azacitidine and gemcitabine, EGF Pathway Inhibitors such as sunitinib, tyrphostin 46, imatinib, EKB-569, sorafenib, erlotinib, pazopanib, gefitinib, and lapatinib, vinca alkaloids such as vinblastine, vincristine, vindesine, and vinorelbine, cyclin dependent kinase inhibitors such as olomoucine, purvalanol B, roascovitine, indirubin, kenpaullone, purvalanol A and indirubin-3′-monooxime, proteasome inhibitors such as aclacinomycin A, gliotoxin, bortezomib, carfilzomib and ixazomib, platinum-based agents including carboplatin, cisplatin, and oxaliplatin, mTor inhibitors such as rapamycin, ridaforolimus, temsirolimus and SDZ-RAD, anthracyclines such as daunorubicin, doxorubicin, epirubicin, idarubicin, mitoxantrone, and valrubicin, topoisomerase inhibitor such as topotecan, irinotecan, etoposide, teniposide, and camptothecin, taxanes such as paclitaxel, docetaxel, larotaxel, and cabazitaxel, epothilones such as ixabepilone, epothilone B, epothilone D, BMS310705, dehydelone, and ZK-Epothilone (ZK-EPO), antibiotics such as actinomycin, plicamycin, daptomycin bleomycin, hydroxyurea, and mitomycin, immunomodulators including lenalidomide and thalidomide, HSP90 inhibitors like geldanamycin, anti-androgens including nilutamide and bicalutamide, antiestrogens such as tamoxifen, toremifene, letrozole, testolactone, anastrozole, bicalutamide, exemestane, flutamide, fulvestrant, and raloxifene, CSF-1R inhibitors such as GW2580.

In other cases, the pharmaceutically active agent can include a biological macromolecule, for instance a therapeutic protein, monoclonal antibodies, vaccine, RNA or DNA. Exemplary vaccines include those against measles, mumps, rubella, diphtheria, typhus, rotavirus, hepatitis, influenza, herpes simplex, malaria, gonorrhea, HIV, and coronavirus.

The shell, or capsule, material can include one or more biocompatible, biodegradable polymers. As used herein, a biocompatible, biodegradeable polymer is a polymer which can be broken down in vivo to monomer and/or oligomer fragments, wherein the monomeric or oligomeric fragments do not provoke an immune response, are not toxic, and can be easily excreted. Exemplary biocompatible, biodegradeable polymers include poly(ethylene glycols) polyesters, mixed polyesters, for instance PLGA, polyanhydrides, mixed polyanhydrides, poly(ester)anhydrides, polysaccharides, polyphosphazenes, and copolymers. In some embodiments, the biocompatible, biodegradable polymer is sufficiently hydrophobic to control the release of the pharmaceutically active agent. The capsule polymer can have a contact angle greater than about 90°, greater than about 100°, greater than about 110°, greater than about 120° greater than about 130°, greater than about 140° greater than about 150°, or greater than about 160°. In some embodiments, the capsule polymer can have a contact angle between about 90-150°, between about 100-150°, between about 110-150°, between about 120-150°, or between about 125-150°. Generally, the core polymer, when present, can be hydrophilic, and can be water soluble such that it degrades/dissolves within 3 hours, within 2 hours, within 1 hour or with 30 minutes of being immersed in water.

The biocompatible, biodegradeable polymer can have an average molecular weight that is no greater than 100,000 Da, no greater than 50,000 Da, no greater than 40,000 Da, no greater than 30,000 Da, no greater than 25,000 Da, no greater than 20,000 Da, no greater than 15,000 Da, no greater than 12,500 Da, no greater than 10,000 Da, no greater than 7,500 Da, no greater than 5,000 Da, or no greater than 2,500 Da. In some embodiments, the biocompatible, biodegradeable polymer can have an average molecular weight that is between 2,500 Da and 100,000 Da, between 2,500 Da and 50,000 Da, between 2,500 Da and 40,000 Da, between 2,500 Da and 30,000 Da, between 2,500 Da and 25,000 Da, between 2,500 Da and 20,000 Da, between 5,000 Da and 100,000 Da, between 5,000 Da and 50,000 Da, between 5,000 Da and 40,000 Da, between 5,000 Da and 30,000 Da, between 5,000 Da and 25,000 Da, between 5,000 Da and 20,000 Da, 10,000 Da and 100,000 Da, between 10,000 Da and 10,000 Da, between 10,000 Da and 40,000 Da, between 10,000 Da and 30,000 Da, between 10,000 Da and 25,000 Da, or between 10,000 Da and 20,000 Da.

In certain embodiments the biocompatible, biodegradable polymer can include one or more of poly(lactic-co-glycolic) acid (“PLGA”), polycaprolactone, polylactide, polyglycolide, polyhydroxybutyric acid, poly(sebacic acid), poly[1,6-bis(p-carboxyphenoxy)hexane], and mixtures thereof.

In certain cases, polycaprolactone can be used in combination with other polymeric systems. Suitable other systems include poly(ethylene glycols) (“PEG”), and PEG copolymers. Exemplary copolymers include polycaprolactone-poly(ethylene glycol), which may further be appended with a functional group such as an amino, thiol, carboxylate and the like. Such functional groups can be used to covalently append biomarkers, dyes, and targeting factors to the encapsulated composition. An example system includes PCL/PCL-PEG-NH₂.

The nanocapsules may be prepared using a voltage-switched coaxial electrospraying process.

Generally, to prepare nanocapsules, a pharmaceutically active agent is dissolved in a first solvent, and a biodegradable, biocompatible polymer is dissolved in a second solvent. The first and second solvents should be capable of dissolving the pharmaceutically active agent and biodegradable, biocompatible polymer, respectively. In comparison with previous processes, the solvent systems can be either miscible or immiscible with each other. The solvent systems can include other excipients, for instance stabilizers, surfactants, antioxidants, and the like. In some embodiments, the first solvent does not contain any of the biocompatible, biodegradeable polymer, and the second solvent does not contain any of the pharmaceutically active agent.

Suitable solvents include aprotic solvents like dimethylsulfoxide (DMSO), halogenated hydrocarbons like chloroform and methylene chloride, ethers like tetrahydrofuran (THF) and diethylether, carbonyl- or nitrile-containing compounds like dimethylformamide (DMF), acetone, acetonitrile, ethyl acetate, and the like. Suitable solvents can also include protic solvents such as water, organic acids like formic acid, acetic acid, propionic acid, trichloroacetic acid, chloroacetic acid, trifluoroacetic acid and the like, or alcohols like methanol, ethanol, ethylene glycol, glycerol, isopropanol, and n-propanol. In certain embodiments, volatile solvents can be used. Exemplary volatile solvents include methanol, ethanol, dichloromethane, acetone, diethyl ether, ethyl acetate and the like. In certain embodiments, the solvent can include an organic acid like formic acid or acetic acid to assist in the dissolution and stability of the compound. Organic acids can be added in an amount of from 0.01-1.0% (v/v), 0.05-1.0% (v/v), 0.05-0.5% (v/v), or 0.05-0.25% (v/v).

In some embodiments, either the first or second solvent can be a mixture of two or more solvents. In some embodiments, the solvent can be a mixture of at least one organic acid and at least one apolar solvent. The ratio (v/v) of organic acid to apolar solvent can be from 1:1 to 99:1, 2:1 to 99:1, 3:1 to 99:1, 4:1 to 99:1, 5:1 to 99:1, 7.5:1 to 99:1, 10:1 to 12.5:1, 15:1 to 99:1, or 20:1 to 99:1. In certain embodiments, the ratio (v/v) of organic acid to apolar solvent can be at least 85:15, 87.5:1, 90:10, 92.5:7.5, 95:5, 97.2:2.5, 98:2 or 99:1. Preferred apolar solvents for combination with the organic acid include halogenated hydrocarbons. Preferred organic acids include formic acid, acetic acid and mixtures thereof. When the organic acid is a mixture of formic acid and acetic acid, the ratio (v/v) can be from 75:25 to 25:75, 60:40 to 40:60, or 50:50. When the first or second solvent includes an organic acid as described above, the other solvent typically contains an aprotic solvent immiscible with the organic acid-containing system. Suitable such solvents include DMF, DMSO, methylene chloride, alkanes like cyclohexane and heptane, and aromatics such as toluene and xylene.

The pharmaceutically active agent can be dissolved in the first solvent, for instance at a concentration from about 1-100 mg/ml, about 5-100 mg/ml, about 10-100 mg/ml, about 25-100 mg/ml, or about 25-75 mg/ml. The biocompatible, biodegradeable polymer can be dissolved in the second solvent at a concentration from about 1-500 mg/ml, 10-500 mg/ml, 25-500 mg/ml, 25-400 mg/ml, 25-300 mg/ml, 25-250 mg/ml, 50-250 mg/ml, 100-250 mg/ml, or 100-200 mg/ml.

Coaxial electrospraying can be conducted using a concentric spinneret nozzle having a core channel through which the core solution is electrosprayed and a capsule channel through which the capsule solution is electrosprayed. An outlet of the capsule channel and an outlet of the core channel are concentrically arranged, and the outlet of the capsule channel is spaced radially outwardly from the outlet of the core channel. The nozzle may include a needle as the core channel, and the needle may have a gauge from 15-34, from 15-30, from 20-30, or from 25-30. In some embodiments, the needle may have a gauge of at least 10, at least 15, at least 20, at least 25, or at least 30. The capsule outlet can have an inner diameter that is no more than 0.2 mm, 0.3 mm, 0.4 mm, 0.5 mm, 0.6 mm, 0.7 mm, 0.8 mm, 0.9 mm, 1.0 mm, 1.2 mm, 1.4 mm, 1.6 mm, 1.8 mm or 2.0 mm. In some embodiments, the capsule outlet can have an inner diameter that is at least about 120%, 140%, 160%, 180%, 200%, 250%, 300%, 400%, or 500% the outer diameter of the needle at the core outlet. In some embodiments, the capsule outlet can have an inner diameter that is between about 120-500%, between about 150-400%, between about 150-300%, or between about 150-250% the outer diameter of the needle at the core outlet.

In some embodiments, the flow rate of the core solution through the spinneret can be at least 0.05 ml/hr, at least 0.10 ml/hr, at least 0.15 ml/hr, at least 0.20 ml/hr, at least 0.25 ml/hr, at least 0.30 ml/hr, at least 0.35 ml/hr, at least 0.40 ml/hr, at least 0.45 ml/hr, or at least 0.50 ml/hr. In some embodiments, the flow rate of the core solution is no more than 0.05 ml/hr, no more than 0.10 ml/hr, no more than 0.15 ml/hr, no more than 0.20 ml/hr, no more than 0.25 ml/hr, no more than 0.50 ml/hr, no more than 0.75 ml/hr, or no more than 1.0 ml/hr. In some embodiments, the flow rate of the core solution through the spinneret can be between 0.05 ml/hr and 1.0 ml/hr, between 0.10 ml/hr and 1.0 ml/hr, between 0.20 ml/hr and 1.0 ml/hr, between 0.05 ml/hr and 0.50 ml/hr, between 0.05 ml/hr and 0.40 ml/hr, between 0.05 ml/hr and 0.30 ml/hr, between 0.20 ml/hr and 0.5 ml/hr, between 0.10 ml/hr and 0.30 ml/hr, between 0.5 ml/hr and 1.0 ml/hr, between 0.75 ml/hr and 1.0 ml/hr, or between 0.20 ml/hr and 0.30 ml/hr.

In some embodiments, the flow rate of the capsule solution through the spinneret can be at least 0.10 ml/hr, at least 0.20 ml/hr, at least 0.30 ml/hr, at least 0.40 ml/hr, at least 0.50 ml/hr, at least 0.60 ml/hr, at least 0.70 ml/hr, at least 0.80 ml/hr, at least 1.0 ml/hr, at least 1.25 ml/hr, or at least 1.50 ml/hr. In some embodiments, the flow rate of the capsule solution is no more than 0.10 ml/hr, no more than 0.15 ml/hr, no more than 0.20 ml/hr, no more than 0.25 ml/hr, no more than 0.50 ml/hr, no more than 0.75 ml/hr, no more than 1.0 ml/hr, no more than 1.25 ml/hr, or no more than 1.50 ml/hr. In some embodiments, the flow rate of the core solution through the spinneret can be between 0.10 ml/hr and 1.50 mg/hr, between 0.10 ml/hr and 1.0 ml/hr, between 0.20 ml/hr and 1.0 ml/hr, between 0.10 ml/hr and 0.50 ml/hr, between 0.20 ml/hr and 0.50 ml/hr, between 0.50 ml/hr and 1.0 ml/hr, between 0.75 ml/hr and 1.0 ml/hr, or between 0.25 ml/hr and 0.75 ml/hr.

The applied voltage for the electrospraying can be between 0.1-50 KV, between 1-50 KV, between 5-50 KV, between 5-25 KV, between 10-25 KV, between 1-100 KV, between 10-100 KV, between 10-75 KV, between 10-50 KV, between 10-40 KV, between 15-40 KV, between 15-30 KV, or between 15-25 KV. The distance from tip to collector can be at least 50 mm, at least 75 mm, at least 100 mm, at least 125 mm, at least 150 mm, at least 175 mm, at least 200 mm, at least 250 mm, or at least 300 mm. In some embodiments, the distance from tip to collector can be from 50-300 mm, from 75-250 mm, from 100-250 mm, from 150-250 mm, or from 100-200 mm. Encapsulated drugs may be collected on a plate, for instance a glass collector plate. After electrospraying, the nanocapsules can be immersed in water to remove residual pharmaceutically active agent from the surface of the fibers and capsules.

Nanoparticles of polymer, e.g., PCL/PCL-NH₂-PEG, having a size range of 15-30 nm, 30-50 nm, or 5-100 nm can be prepared. In some cases, the electrospraying can be done with various polymers, for instance those having MW from 2,000-14,000 g/mole.

The nanoencapsulated drugs may be combined with one or more targeting vectors or biomarkers/Fluorescent dye/IR Dye. For instance, when the polymer includes PCL-PEG-NH₂, (2000 MW) the nanoparticles can be coupled to IRdye650 for the proposed targeting therapies. For example to tag with IR Dye 650 NHS, the nanoparticles are dispersed in an PBS/MES Buffer solution (deprotonate the amine group/activated) that is added with appropriate amount of a nanoparticles that is stirred overnight at room temperature to complete the coupling reaction. Stock solutions of concentration 1 mg/1 mL were made of both the PCL-PEG-NH₂ and the IR dye. The nanocapsules are recovered using a high-speed centrifuge, and vacuum drying and or through size dependent dialysis tubes, benzoylated (2000MWCO) and then rinsed thoroughly using deionized water to remove any impurities for the following cell studies.

Nanoencapsulated drugs can be directly obtained through a pulsed, co-axial electrospraying process. In such embodiments, a core solution and a capsule solution are coaxially electrosprayed through a plate. The plate defines a core channel having a core outlet and a capsule channel having a capsule outlet. For example, in some implementations, the plate includes a coaxial spinneret nozzle, such as described above, that defines the core outlet and capsule outlet. The capsule outlet is coaxial with the core outlet and is disposed radially outwardly from the core outlet. The core solution comprises at least one solvent and at least one active agent, and the capsule solution comprises at least one solvent and at least one polymer. Coaxial electrospraying includes (1) applying a pulsating voltage at a frequency to the plate, wherein the pulsating voltage fluctuates between a minimum pulsating voltage and a maximum pulsating voltage, wherein the minimum pulsating voltage is 0 KV or greater and the maximum pulsating voltage is greater than 0 KV and the minimum pulsating voltage; and (2) applying a constant voltage to the plate, the constant voltage being greater than 0 KV. The total maximum applied voltage to the plate is the sum of the maximum pulsating voltage and the constant voltage, and the total minimum applied voltage to the plate is the sum of the minimum pulsating voltage and the constant voltage. The pulsating voltage and the constant voltage are applied while the core solution and the capsule solution are flowing through the core channel and the capsule channel, respectively. The formed nanocapsules are collected on a grounded collection plate spaced apart from the plate.

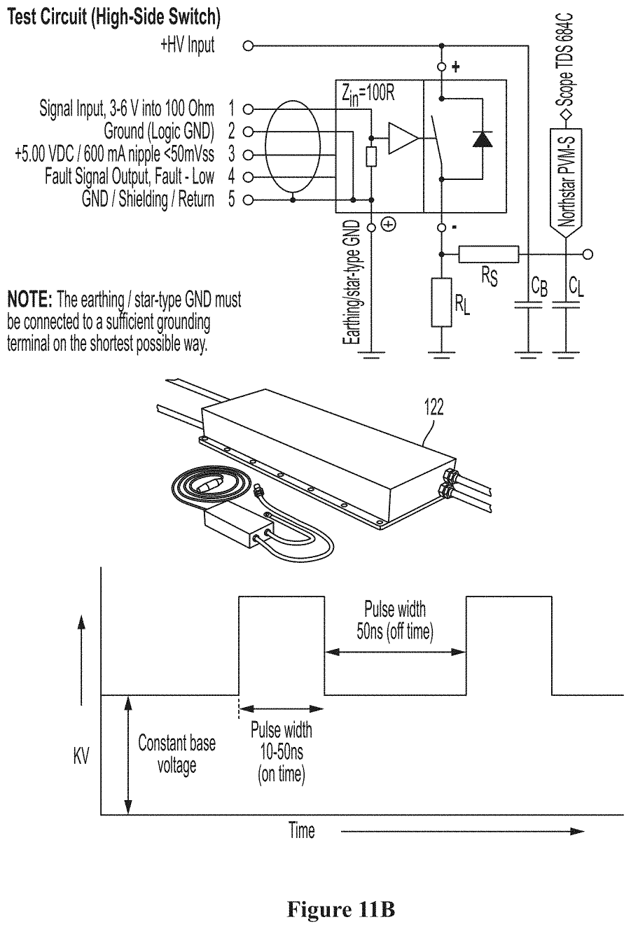

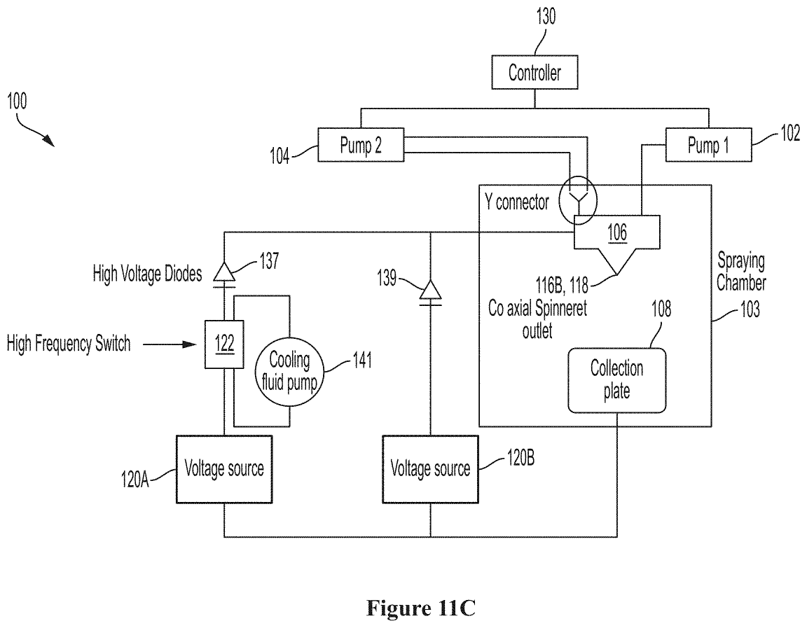

FIGS. 10A-11C illustrate a coaxial electrospraying system 100 for forming nanocapsules according to the process described above, according to one embodiment. The system 100 includes a core solution pump 104, a capsule solution pump 102, a plate 106, a collector 108, and at least one voltage source, shown here as dual voltages sources 120A, 120B that provide a pulsating voltage (via transistor switch 122 and diode 137) and a constant voltage (via diode 139). However, in other embodiments, the constant voltage and the pulsating voltage may be provided in other configurations.

As shown in FIGS. 10A and 11A, core solution pump 104 is in fluid communication with first and second core solutions 112a, 112b. Capsule solution pump 102 is in fluid communication with capsule solution 114. The pumps 102, 104 are positive displacement pumps that provide a constant flow of the solutions. For example, the pumps 102, 104 may include syringe pumps. Each pump may actuate one or more syringes. In the embodiment shown in FIGS. 10A and 11A, a first syringe includes first core solution 112a, and a second syringe includes second core solution 112b. These syringes are actuated by pump 104. And, the syringe that includes capsule solution 114 is actuated by pump 102. However, in other embodiments, the system may include one or more core solutions, two or more capsule solutions, two or more core solution pumps, and/or two or more capsule solution pumps.

As shown in FIGS. 10B and 10C, the plate 106 includes a concentric nozzle 105 that defines a core channel 116 and a capsule channel 118. The core channel 116 has a core outlet 116a and a core inlet 116b. As noted above, a needle may define the core channel 116, such as the needles described above. The capsule channel 118 has a capsule outlet 118a and a capsule inlet 118b. The core outlet 116a and capsule outlet 118a are coaxial, and the capsule outlet 118a is disposed radially outwardly of the core outlet 116a. In other words, the capsule outlet 118a is spaced apart from the core outlet 116a in the radial outward direction. As shown in FIGS. 10B and 10C, the nozzle 105 is separately formed from the plate 106 and is coupled to the plate 106 and is in electrical communication therewith. In other words, current flowing through the plate 106 flows through the nozzle 105.

The core outlet 116a is in fluid communication with the core solution pump 104, and the capsule outlet 118a is in fluid communication with the capsule solution pump 102. For example, in the implementation shown in FIGS. 10A and 11A, the system 100 includes a core solution manifold 126, such as a Y-connector. The core solution manifold 126 has two inlet openings 126a1 and 126a2 coupled to conduits that extend from the end of each syringe of the core solution pump 104 and a single exit opening 126b that is coupled to a conduit that extends from the inlet 116b of the core channel 116. The first core solution 112a and the second core solution 112b are received by the inlet openings 126a1, 126a2, respectively, and are combined in the core solution manifold 126 prior to flowing to the core channel 116 of the plate 106. In some embodiments, the first core solution is immiscible in the second core solution. In some embodiments, the inlet openings of the manifold are directly coupled to the syringes of the core solution pump and/or the exit opening of the manifold is directly coupled to the core inlet of the core channel. In other embodiments, the core solution manifold may include more than two inlet openings. For example, the core solution manifold may have inlet openings corresponding to and for receiving each core solution. And, in some embodiments in which there is only one core solution to be included in the nanocapsules, the system does not include the manifold or only utilizes the manifold for the one core solution.

The system 100 has one capsule solution, but in other embodiments, the system may include more than one capsule solution. In certain embodiments, such a system includes a capsule solution manifold disposed between the capsule inlet and the capsule solution pump(s). The capsule solution manifold has inlet openings corresponding to and for receiving each capsule solution. The capsule solution manifold also has a single exit opening, and the single exit opening is in fluid communication with the capsule outlet. The two or more capsule solutions are combined in the capsule solution manifold prior to flowing to the capsule channel of the plate. In some embodiments, the two or more capsule solutions are immiscible. In some embodiments, the inlet openings of the capsule solution manifold are coupled to the capsule solution pump(s) (e.g., to syringes of the capsule solution pump(s)) by conduits that extend between the inlet openings of the capsule solution manifold and the capsule solution pump(s), and a conduit extends between the single exit opening of the manifold and the capsule inlet. In other embodiments, the inlet openings of the manifold are directly coupled to the capsule solution pump(s) and/or the exit opening of the manifold is directly coupled to the capsule inlet. In one embodiment, the capsule solution manifold may include a Y-connector, such as Y-connector 126 shown in FIG. 10A, when two capsule solutions are being combined prior to flowing to the capsule channel of the plate. And, in some embodiments in which there is only one capsule solution to be included in the nanocapsules, the system does not include the manifold or only utilizes the manifold for the one capsule solution.

In addition, the system 100 includes a controller 130 for controlling a flow rate of the core solution pump 104 and a flow rate of the capsule solution pump 102. In some implementations, the flow rates are controlled such that the flow rate of the capsule solution 114 into the capsule inlet 118b is greater than the flow rate of the core solutions 112a, 112b through the core inlet 116b. In addition, in some implementations, the flow rates of the core solutions 112a, 112b may be controlled such that the flow rate of one of the core solutions is greater than the flow rate of the other core solution. For example, in one implementation, the flow rates of the core solutions 112a, 112b may be different.

The core solutions 112a, 112b include at least one pharmaceutically active agent, e.g., chemotherapeutic agents or contraceptives. Examples of suitable chemotherapeutic agents are described above. For example, in some embodiments, core solution 112a may be a taxane as the chemotherapeutic agent, such as paclitaxel. And, core solution 112b may include a tumor microenvironment (TME) altering agent, such as CSF-1R inhibitors, such as GW2580. In addition, in other embodiments, the core solutions may include proteins, fluorescent dyes, and/or biomarkers.

The capsule solution 114 includes one or more biocompatible, biodegradable polymers, such as those described above. For example, the biocompatible, biodegradable polymer comprises one or more polyester, mixed polyester, polyanhydride, mixed polyanhydride, poly(ester)anhydride, polysaccharide, polyphosphazene or polyphosphoester. As another example, the biocompatible, biodegradable polymer comprises PLGA, polycaprolactone, polylactide, polyglycolide, polyhydroxybutyric acid, poly(sebacic acid), poly[1,6-bis(p-carboxyphenoxy)hexane], polyvinylpyrrolidone, polyvinyl alcohol, poly(ethylene oxide), poly(propylene oxide), copolymers thereof, or a mixture thereof. In some embodiments, the capsule solution 114 can include one or more pharmaceutically active agents, proteins, dyes or biomarkers as described above. In additional embodiments, capsule solution 114 can include an amine functionalized polymer (e.g., PCL-PEG-NH₂) for conjugation to one or more targeting vectors or biomarkers/Fluorescent dye/IR Dye.

As shown in FIGS. 11A, 11B and 11C, dual voltage sources 120A, 120B are electrically coupled to the plate 106. The voltage source 120B applies a constant voltage, and a voltage source 120A, in series with transistor switch 122, provides a pulsed voltage to the plate 106. The constant voltage is greater than 0 KV. The pulsed voltage fluctuates between a minimum pulsating voltage and a maximum pulsating voltage. The minimum pulsating voltage is 0 KV or greater, and the maximum pulsating voltage is greater than 0 KV and the minimum pulsating voltage. In some embodiments, the voltage source 120A includes a transistor switch 122, such as shown in FIGS. 11A and 11C. The transistor switch 122 pulses the voltage between the minimum pulsating voltage and the maximum pulsating voltage at a switching frequency. For example, in some embodiments, the maximum pulsating voltage is less than or equal to 50 KV and the minimum pulsating voltage is 0 KV. In certain embodiments, the maximum pulsating voltage is less than or equal to 40 KV, less than or equal to 30 KV, less than or equal to 20 KV. The selection of the maximum pulsating voltage may depend on the equipment used in the system 100, the solvents and/or solutes used, molecular weights of the materials used in the system, loading concentrations desired, and other factors determine by selected process variables in any given situation. And, the pulse width for applying the maximum and minimum pulsating voltages may be between 10-50 ns (nanoseconds).

The constant voltage selected to be applied can be between 10 KV and 40 KV, according to some implementations. Thus, in certain non-limiting implementations, the total maximum applied voltage to the plate fluctuates between voltages equal to or greater than 10 KV and equal to or less than 90 KV.

As shown in FIGS. 11A, 11B and 11C, the pulsating voltage emanating from the transistor switch 122 and the constant voltage emanating from the voltage source 120B are applied to the plate 106 along parallel voltage lines 124, 125, respectively. As illustrated in the example of FIG. 10C, in at least one embodiment, the system 100 incorporates two separate voltage sources 120A and 120B that operate in the ranges described herein for applying the pulsating voltage on line 124 and the constant voltage on line 125 to the place 106. The first voltage source 120A is connected in series to the transistor switch 122 to maintain the selected pulsating voltage at line 124 connected to the plate 106, and the second voltage source 120B provides, to the plate 106, the constant voltage on line 125 in parallel to the pulsating voltage line 124. FIGS. 11A-11C disclose other components present in the system 100 described herein, including the diodes 137, 139 regulating the pulsating voltage and the constant voltage, cooling fluid pump 141 for keeping the transistor switch and associated electronics at safe temperatures that are specified for the equipment at hand, and the controller 130 programmed to provide pump output levels according to specifications for the use at hand. These electronics ensure that the spraying chamber 103 operates at a desired rate and with a usable output.

The power dissipation in the high-frequency switching device (e.g., transistor switch 122) in the form of heat depends on the combined effect of the level of the applied voltage (Volt/Kilo Volt) and the switching frequency (Hertz/Kilo Hertz/MegaHertz). The switching device is integrated with a cooling system (e.g., a pump and cooling fluid circuit) to remove the heat from the switching device. To keep the heat dissipation within the capacity of the cooling system, the voltage and the switching frequency are used in inversely proportional relation. For example, if a higher level of voltage (such 90 KV) is used, the switching frequency is decreased so that power dissipation in the form of heat does not increase. On the other hand, if a higher switching frequency is used, the voltage can be decreased to keep the switching device within the operating level. The voltage and the switching frequency level can be adjusted based on the capacity of the switching device and efficiency of the cooling system. As the switching frequency increases, the pulse width decreases.

And, in some non-limiting embodiments, a single voltage source may be used to provide the constant and pulsating voltages. The single voltage source may include high voltage switching devices, such as transistor switches, embodied in power semiconductor stacks of solid-state equipment.

In other embodiments, the voltage source includes two voltage sources—one voltage source for applying the constant voltage and one voltage source for applying the pulsed voltage.

The collector 108 is a plate that faces an outlet side of the plate 106 and is spaced apart therefrom. The collector plate 108 is grounded relative to the voltage source 120.

As noted above, the process may also include sonicating one or more of the core solution, capsule solution, or precursor solutions thereof, prior to co-axial electrospraying. Sonication may not be necessary in some embodiments, depending on the properties of the solutions, their molecular weight, and the morphology desired in the end product.

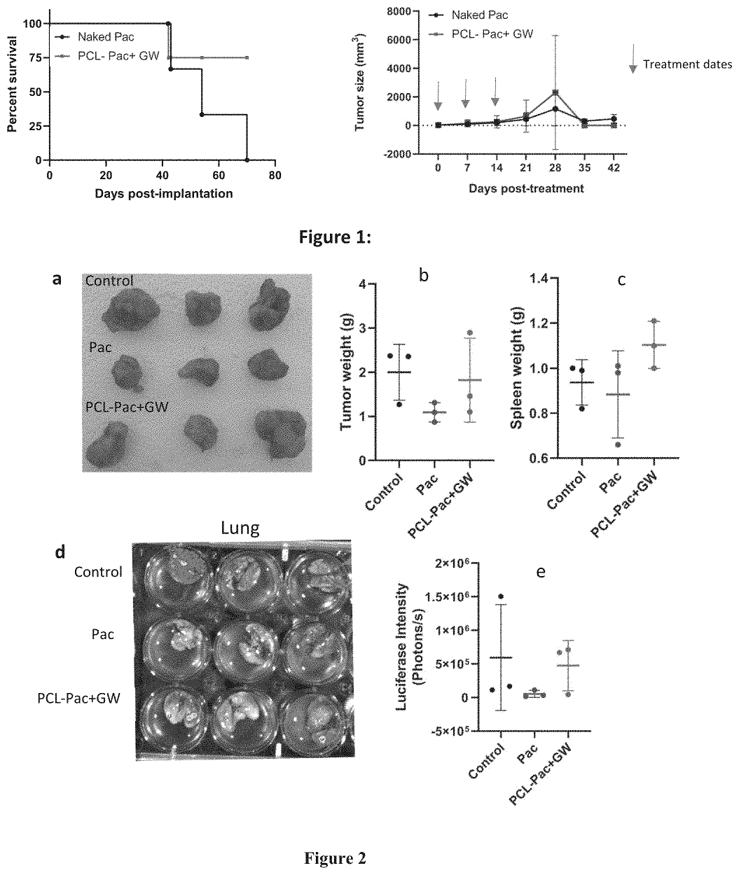

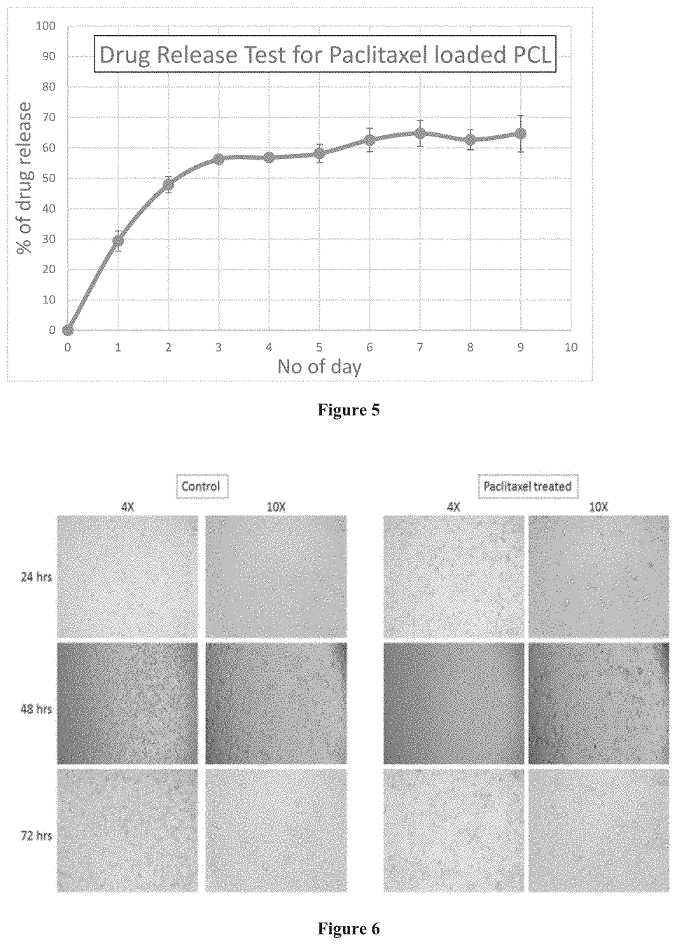

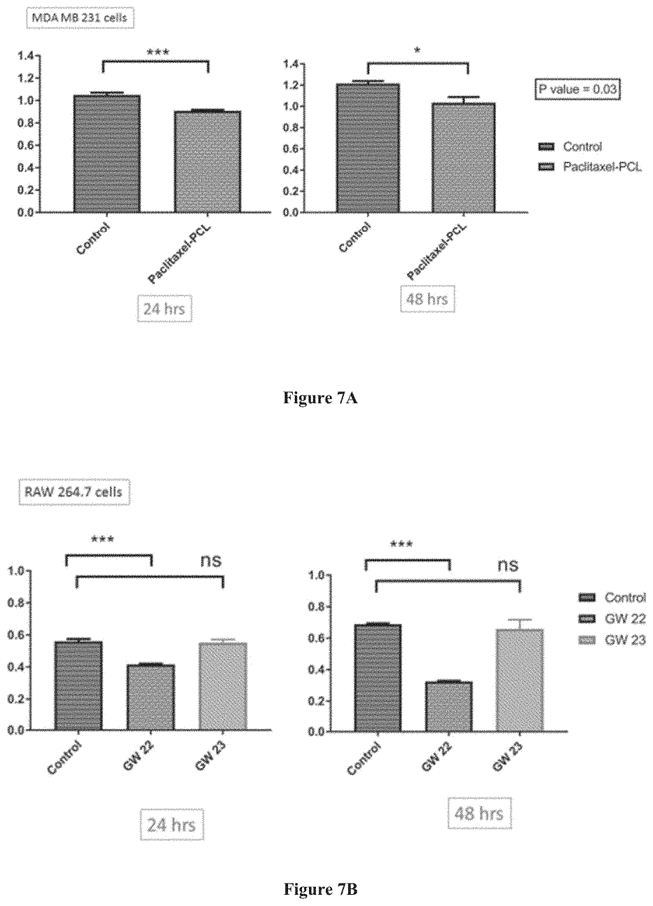

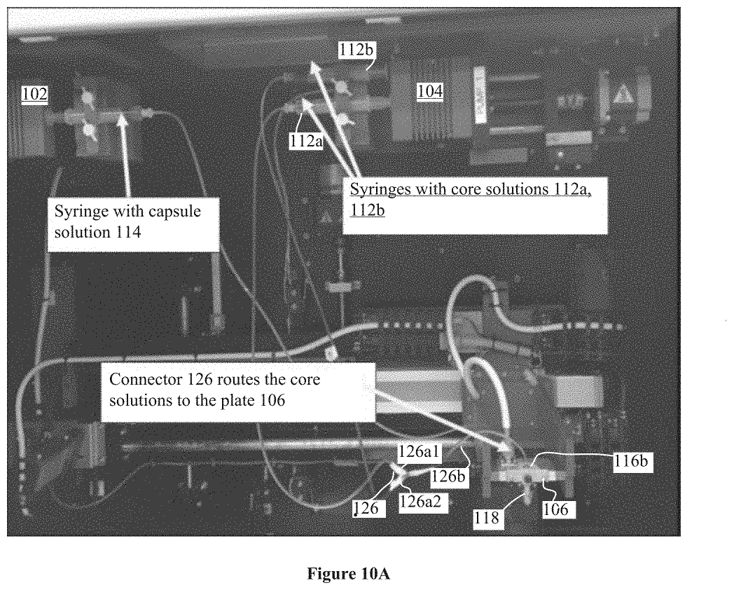

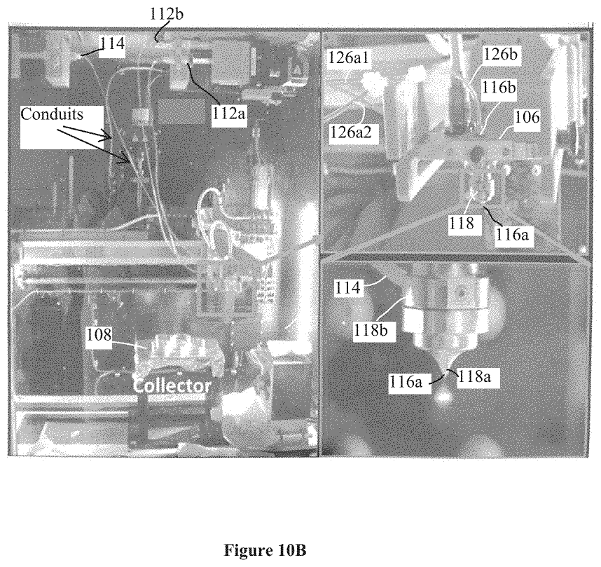

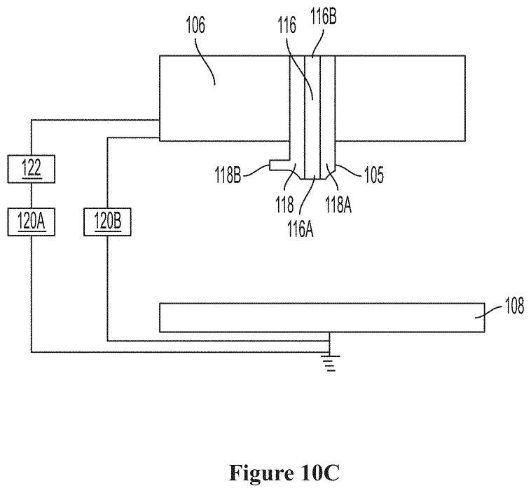

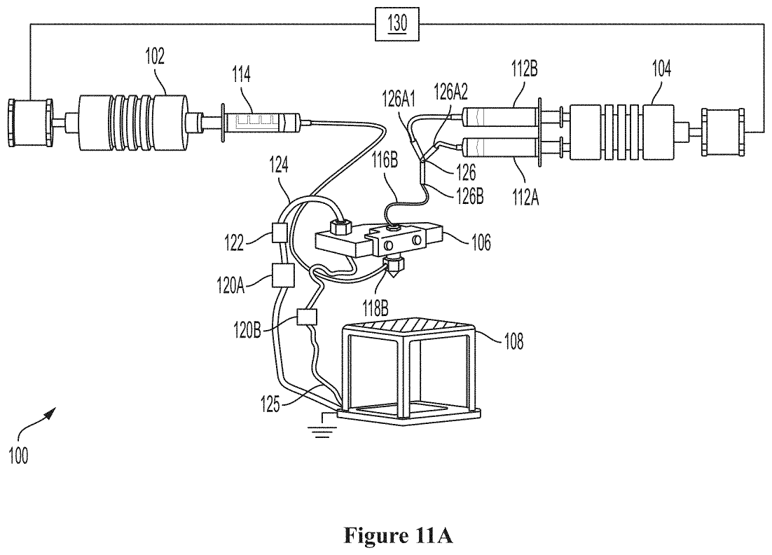

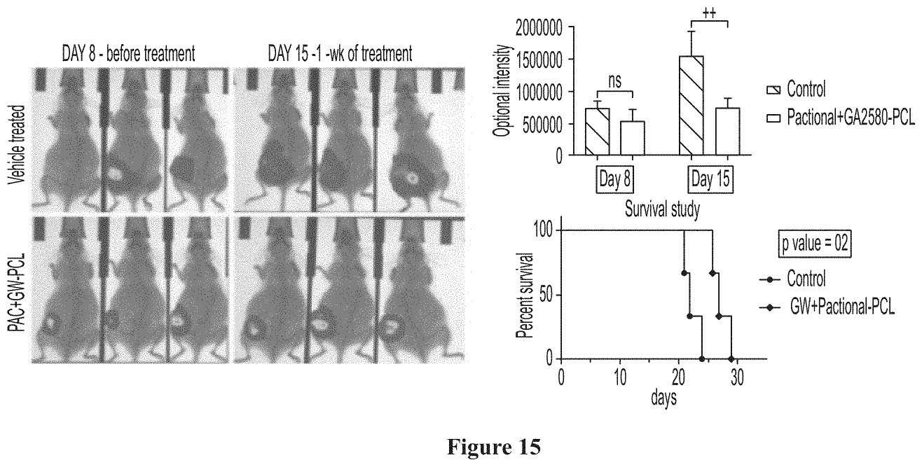

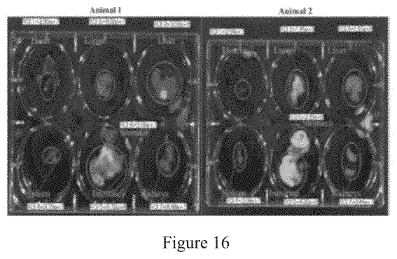

Examples of nanoencapsulated drugs are described below in relation to FIGS. 1-9 and 12-16.

In the above-described embodiments, constant and pulsating voltage are applied to the plate to form nanocapsules. However, in other embodiments, systems and methods for forming nanocapsules may apply constant voltage only or may apply pulsating voltage only.

### EXAMPLES

The following examples are set forth below to illustrate the methods and results according to the disclosed subject matter. These examples are not intended to be inclusive of all aspects of the subject matter disclosed herein, but rather to illustrate representative methods, compositions, and results. These examples are not intended to exclude equivalents and variations of the present invention, which are apparent to one skilled in the art.

Efforts have been made to ensure accuracy with respect to numbers (e.g., amounts, temperature, etc.) but some errors and deviations should be accounted for. Unless indicated otherwise, parts are parts by weight, temperature is in ° C. or is at ambient temperature, and pressure is at or near atmospheric. There are numerous variations and combinations of reaction conditions, e.g., component concentrations, temperatures, pressures, and other reaction ranges and conditions that can be used to optimize the product purity and yield obtained from the described process. Only reasonable and routine experimentation is required to optimize such process conditions.

### Example 1: Core/Shell Nanocapsules

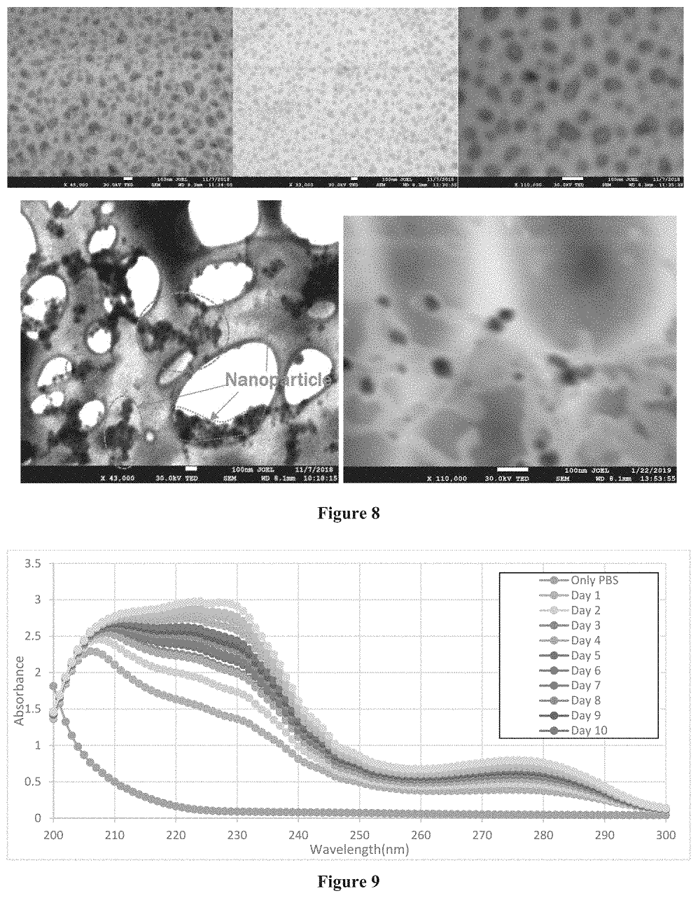

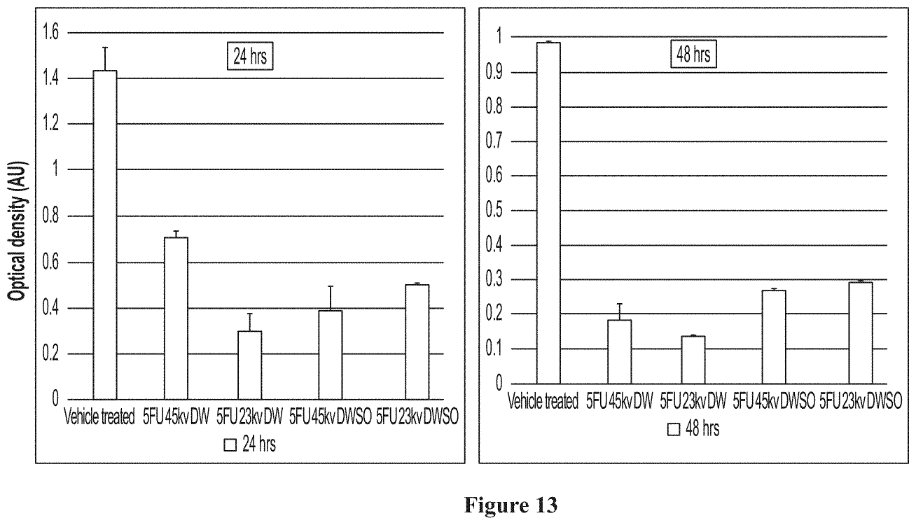

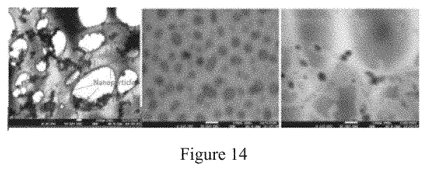

Crystallinity of anticancer drugs and capability of formation drug nanocrystals was investigated using electrospraying. Stability of anticancer drugs in a different solvent which also possess high electric conductivity and volatility required to get good electrospraying as well as stability of drug nanocrystals in media plays an important role. Several anticancer drugs such as FU, paclitaxel, and cyclophosphamide has been successfully electrosprayed into submicron size nanocrystals using very high voltage (as high as 46 KV). The efficacy of the cancer drug nanocrystals was tested. Cell viability was measured using a Cell Proliferation Reagent WST-1 (MilliporeSigma, USA). As shown in FIG. 13, significant decrease of cell viability was observed with all the drug nanocrystals sample compared to control (vehicle treated) in both 24 hours and 48 hours. We produced PCL encapsulated single (paclitaxel) and combination (paclitaxel+GW2580) nanocapsules within the size range of <100 nm, which is shown in FIG. 5. PCL—paclitaxel nanocapsules, and PCL-GW2580 as single drug system was produced where drug dose loading was varied between 4% to as high as 25%. For multidrug system the different drug solution feeding mechanism was developed to produce multidrug nanocapsules with varying drug rations between them. When nanocapsules with simultaneous two cancer drugs such as paclitaxel and GW2580 were produced, different ratios between the drugs were maintained. For example, the ratios of paclitaxel and GW2580 were 1:1; 1:2; 1:4 and 1:8 in the produced multidrug nanocapsules to achieve different dosing ratios. The combined drug polymer ration also was varied in the system. The combined drug in the core was possible to vary between 7 to 25 wt % of the total sample. The TEM image in FIG. 14 shows the PCL encapsulated multi drug (paclitaxel+GW2580) nanoparticles. The size distribution of the produced nanoparticles was less than 100 nm even before the osmosis filtering. The total drug in the core was about 25% and the rest was the sheath biopolymer PCL. All these injectable nanocapsules were produced using electrospraying techniques with innovative feeding and spinneret system as shown in FIG. 10A. The synthesis of the drug and the polymer solution were performed using ultrasonic materials processing technique with compatible solvent systems.

The synthesis and fabrication process was performed in two different methods. The first process included electrospraying of single and combined drug with sheath polymers PCL and PCL-PEG-NH₂ using constant DC voltage. The second process included co-axial electrospraying with high frequency switching (in KHz) DC voltage (e.g., a maximum pulsating voltage on line 124 of 20-50 KV applied in parallel to the plate 106 in addition to the constant voltage on line 125) to create a core (drugs) and sheath (PCL/PCL-PEG-NH₂) nanocapsules with in the size range of 10-100 nm.

In the first approach, electrospraying of single and multidrug nanocapsules was performed using constant DC voltage. Based upon the required comparative dose of the drugs for in-vivo analysis, concentration of paclitaxel and GW2580 was determined. Paclitaxel was dissolved in formic acid with a concentration of 4.4 mg/mL. A homogeneous solution was prepared using magnetic stirrer in a light protective environment (by covering the beaker with aluminum foil) as paclitaxel has very high light sensitivity. GW-2580 was dissolved in dimethyl sulfoxide (DMSO) with a concentration of 24.34 mg/mL. Both of these solutions were used as core solutions in the electrospraying process shown in FIGS. 10A-11C.

Biocompatible polymer, polycaprolactone (PCL) was dissolved in a combination of three solvents with a concentration of 32.86 mg/mL. A three-component solvent system consisting of formic acid (FA), acetic acid (AA) and trifluoroethanol (TFE) with a volume ratio of 9:9:1. External agitation such as magnetic stirring was used for 1 hour with a speed of 700-800 rpm to completely dissolve the PCL into solvent system. Three solvent system was used to lower the conductivity and volatility of the capsule solution compared to the core solution. As PCL has high molecular weight, it has high polymer chain entanglement. Systematic high-power (750 watt capacity with 20 KHz) ultrasonic materials processing unit was used to sonicate the PCL solution. It is required to homogeneously disperse the polymer chain, which is eventually targeted to break during the electrospraying process. A Vibra cell Sonics ultrasonic processor with 13 mm probe was used to ensure proper acoustic wave propagation in the solution along all directions uniformly. The sonication parameter is: Amplitude 30%; Pulse frequency: 50 sec ON and 10 sec OFF; Temperature Set: 47° C.; Total Sonication Time: 13 hours. Maintaining the temperature during sonication prevents any unwanted evaporation of the solution during operation. A temperature feedback system was used to maintain the temperature of solution at around 47° C. Any further temperature rise during the sonication process caused automatic termination which ensures proper temperature control.

The first and second electrospraying processes were used to process both single and multi-drug nanocapsules. The plate 106 shown in FIGS. 10A and 10B includes an ultrafine coaxial spinneret, which is a concentric nozzle 105 used for creating a coaxial stream of core drug solution and capsule (or sheath) polymer solution. A 27-gauge needle defining the core channel 116 was placed into the capsule channel 118 of the nozzle 105 such that the core outlet 116a was concentric with the capsule outlet 118a. The inner diameter of the capsule outlet was 0.8 mm. Solutions were pumped to the spinneret using two separate high precision syringe pumps and 10 ml NORM-JECT latex free syringes. In the case of a multi drug system, two separate core drug solutions were sent as the core solution by pumping the core drug solutions individually with a single core solution pump and using a Y-connector luer lock as the core solution manifold. Capsule polymer solution was pumped as a capsule solution by high precision syringe pump. A separate dehumidifier unit was used to control the humidity of the electrospraying chamber. Relative humidity was maintained around 40 to 45% throughout the electrospraying process. The total flow rate (sum of the core and capsule flow rate) was controlled to achieve continuous electrospraying and encapsulated drug nanoparticles. The comparative flow ratio and concentration of the two individual drugs were controlled very precisely to maintain the ratio of the mass flow rate according to the doses. For example, the system is capable to process nanocapsules with multiple drugs with drug weight ratios of 1:1; 1:2; 1:4; 1:8 and more. The weight percentage of the total drug with respect to the polymer can be varied between 4% to 25% depending on the total does required. All the system parameters were optimized to produce nanocapsules with repeatable and uniform morphology and size distribution. Nanocapsules of individual drugs (PCL-paclitaxel and PCL-GW2580) were produced in the similar manner except for using the Y-connector in communication with the core channel. A single core drug solution conduit was directly connected at the core channel inlet of the plate and the capsule polymer solution conduit was directly connected at the capsule channel inlet of the plate. All other parameters were controlled and adjusted according to the drug-polymer ratio for the specific dose needed. The electrospraying of the nanocapsules was conducted in closed chamber of the equipment.

In the second process, nanocapsules of paclitaxel and GW2580 with PCL/PCL-PEG-NH₂ capsule material (10-100 nm) were processed using the system shown in FIGS. 10A and 10B by coaxial electospraying using high frequency switching voltage power supply (e.g., pulsed signal with 10-50 nanosecond pulse width). Carrier polymer PCL/PCL-PEG-NH₂, anticancer drug paclitaxel and GW 2580 were dissolved in two separate solvents to make PCL and drug solution separately. Both solutions were pumped by syringe pumps to the spinneret to form a compound Taylor cone. The compound Taylor cone was drawn from the spinneret tip with the influence of DC high voltage supply and jet breakup was ensured through pulsating voltage (e.g., a pulsating voltage that can range from 20-46 KV and applied in parallel to a constant voltage). If the voltage is interrupted, then instability of the stretched compound fluid jet starts to break which results in formation of spherical beads in the collector. By controlling the switching of the voltage (e.g., on and off at 10-50 ns time steps) continuous core-sheath electrospinning is transformed into discontinuous core-sheath electro-spraying which generates encapsulated ultrafine drug particles rather than fibers. The system is capable generating constant high voltage DC power supply (<50 KV), and a high voltage high frequency switching device/pulse generator, such as the transistor shown in FIG. 11B, is included to control frequency (<100 KHz) and amplitude of the pulsed DC voltage. The resulting output voltage is a high frequency square wave, such as shown in FIG. 11B, with controllable constant base voltage supply. The purpose of the constant base voltage is to hold Taylor cone, and the purpose of the DC square wave (on time) is to draw the compound jet from the Taylor cone. By applying switching voltage (e.g., as low 10 ns duration for the maximum pulsating voltage and then minimum pulsed voltage) a discontinuous pulsating jet of co-axial flow is obtained. The size of hydrodynamic pulsating jet is controlled by controlling pulse magnitude and width of the voltage and the switching frequency. Through this technical approach, doses control are achieved by controlling the drug and polymer concentration ratio during the synthesis phase. Moreover, this method is applicable to wide variety of cancer drugs along with paclitaxel and GW2580.

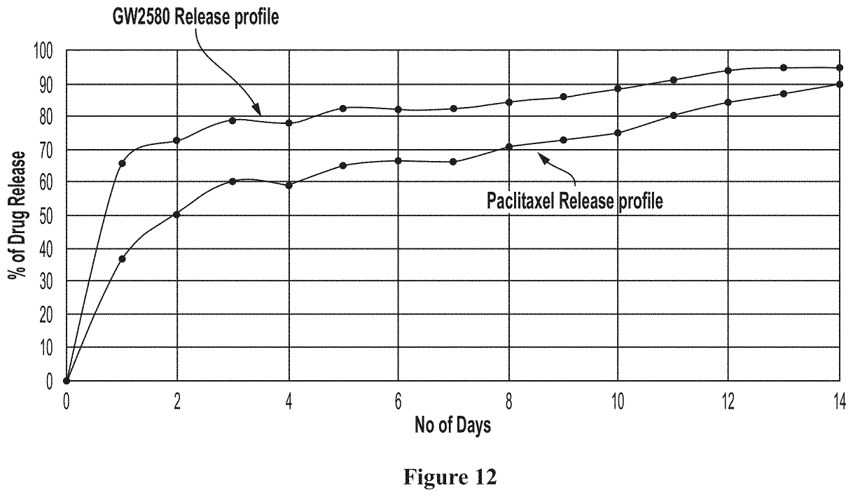

In vitro UV-Vis spectroscopy drug release tests were performed both for single and multidrug system nanocapsules. NPs were dried through vacuum dryer. NPs were then suspended to PBS media and kept in the UV incubator at 37° C. Nanoparticle suspended solution of 50 μL was taken into different microplate for UV-Vis test. UV tests were performed over a wide range of wavelength to detect the presence of drug and its concentration through measuring the absorbance spectra corresponding to a specific drug obtained from the UV-Vis. A calibration curves for the specific drug concentration vs UV-Vis absorbance were performed. The calibration curves against the absorbance were used to determine the actual cumulative drug release at each day percent release of the specific drug were plotted. The drug release tests were conducted for 2-3 weeks. FIG. 12 shows the drug release profile for the combined drug nanocapsules. Approximately 60% to 70% drugs were released within first 2 days, however the rest of the drugs were released over the next 12 days. It indicates a prolonged and sustainable release pattern of the drugs from the nanocapsules.

For the in vivo treatment and survival study, syngeneic luciferase+4T1 cells were orthotopically implanted in the fat pad of 5-6 weeks old female balb/c mice. Tumor growth was monitored every week. In vivo, optical images were obtained every week to keep track of primary tumor and metastasis development by injecting 100 μL of luciferin (3 mg/mL) intraperitoneally followed by the acquisition of bioluminescence signal by spectral AmiX optical imaging system (Spectral instruments imaging, Inc. Tucson, Ariz.). The photon intensity/mm/sec was determined by Amiview software (version 1.6.0). Percentage of paclitaxel and GW2580 was approximately 2.7% and 22.2%, respectively, in the combined drug formulations. Sterile water was added to the formulation followed by sonication to make an injectable solution. Drug formulation was administered into the tumor bearing mice intravenously through tail vein in 100 mL solution at a dose of 20 mg/kg/day for paclitaxel or 160 mg/kg/day for GW2580. Treatment was started on day 8 post-implantation and continue for two weeks (every alternate day, total 6 doses). All the animals tolerated the combined paclitaxel and GW2580. There were no adverse effects, in respect of weight loss and other wellbeing, observed during the therapy. All vehicle treated animals died within 24 days but the animals that received treatments survived for 29 days (FIG. 13). While photon intensity in the tumor on day 8 post-implantation for all the animals (both groups) did not show any significant difference, but on day 15 (after 1 week of treatment) group of animals injected with nanonized combined paclitaxel and GW2580 showed significant decreased of photon intensity compared to that of vehicle treated animals (FIG. 13). Kaplan Myer statistical analysis showed significant survival benefit with the nanocapsules of combined drug of paclitaxel and GW2580.

In addition, electrosprayed PCL-PEG-NH₂ nanoparticles (Mw of 2000) within the size range of 30 to 40 nm were produced, as shown in FIG. 5. PCL-PEG-NH₂ nanoparticles were then tagged with IRDye 650 NHS to assess the biodistribution of the nanoparticles in mice. 100 μl of PCL-PEG-NH₂ nanoparticles at a concentration of 420 mg/L was injected through tail vein. After an hour, the mice were euthanized and liver, lungs, heart, spleen, intestine and kidneys were collected into separate 6-well plates and fluorescent images were acquired. Photon intensity from fluorescence images showed PCL-PEG-NH₂ nanoparticles are well distributed in different organs. Most prominent photon intensity is in gastrointestinal tract and liver and the organ with least photon intensity is heart. Interestingly it showed better biliary excretion than renal excretion.

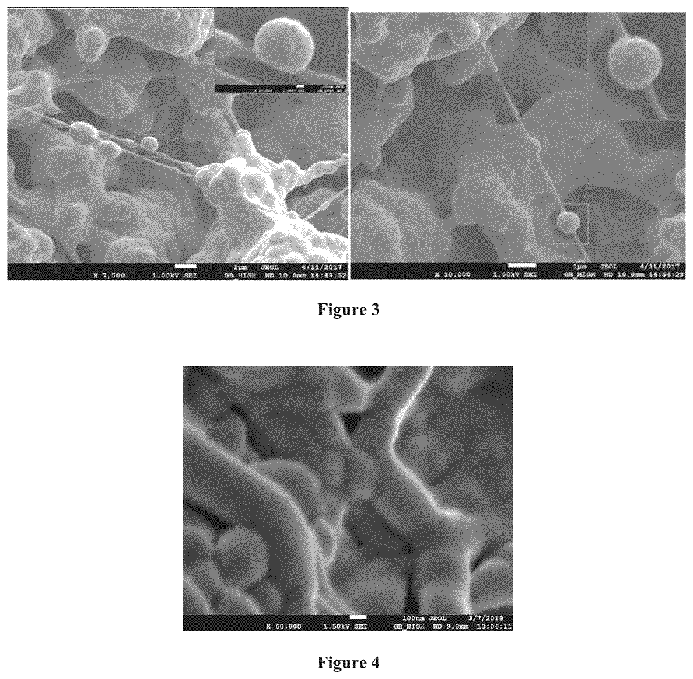

To investigate the efficacy of nanoscale encapsulation of cancer drugs CSF-1R inhibitor (GW2580) and paclitaxel into biocompatible polycaprolactone (PCL) nanocapsules in tumor growth and metastatic outgrowth, we utilized the metastatic 4T1 tumor cells. Murine 4T1 cells were originally isolated from a spontaneous mammary tumor in the BALB/c strain and have been reported to show characteristics of human triple-negative breast cancer (TNBC) subtype. We first implanted 50,000 4T1 tumor cells into mammary fat pad of syngeneic mice and started treatment one-week post-implantation. After 3 doses by injection (one dose/week), the treatment was terminated and mice were followed for tumor growth and survival. In the first set of mice, only one mice in the encapsulated group died within 40 days and the remaining mice were tumor-free after treatment and survived until the time we followed up (70 days). However, all the mice in naked paclitaxel-treated group died within 70 days (FIG. 1), which is longer than the tumor bearing mice that did not have any treatment. Next, we examine the efficacy of encapsulated paclitaxel and GW2580 on metastatic outgrowth of disseminated tumor cells. GW2580 is a selective inhibitor of colony stimulating factor I-receptor (CSFIR), which is a key regulator of myeloid cell proliferation, survival, and differentiation. Inhibition of CSFIR has been shown to alter recruitment and polarization of immunosuppressive myeloid cells. Our group also showed that pre-treatment of mice with CSFIR inhibitor, GW2580 before tumor implantation prevent the recruitment of myeloid cell infiltration into lung, one of the main metastasis targeting organ for breast cancer cells. Second set of mice euthanized to examine metastatic colonization of tumor cells in the lungs 72h after the last injection. IP-injection of naked paclitaxel decreased the tumor weight but this decrease was not significant compared to control or encapsulated treatment. (FIG. 2a-b). Although encapsulated paclitaxel (PCL-Pac+OW) showed a tendency towards increased weight, no significant difference was detected for weight of spleens between groups (FIG. 2c). Next, we performed ex vivo lung imaging and it showed that micro metastatic colonization of tumor cells in the lungs of naked pac-treated animals whereas two of animals in encapsulated pac-treated group showed macro metastasis in same organ. Yet, signal intensity of luciferase was not significantly different between groups (FIG. 2d-e).

### Example 2: Electrospray of TAMS-1 Peptides

TAMS-1 peptides (2 mg) was combined with 32 ml of dimethyl sulfoxide. The solution was mixed with a small magnetic stirrer at 320 rpm for 20 minutes to provide a solution with an estimated concentration of 0.0625 mg/ml. The PCL solution was prepared by combining 1.314 g PCL, 18.94 mL of Acetic Acid, 18.94 mL of Formic Acid, and 1.05 mL Trifluoroethanol to keep a 9:9:1 ratio of AA:FA:TFE. The solution is mixed with a magnetic stirrer at 720 rpm for 30 minutes, or until the PCL looks completely dissolved in the solvents. The PCL solution is then sonicated for a total of 24 hours at a 30% amplitude with a pulse frequency of 50 seconds on, and 10 seconds off. To avoid evaporation of the solution, a temperature probe is put into the solution and set at a temperature of 47° C. to turn the sonicator off if the temperature reaches 47° C. or higher. To avoid high temperatures, the beaker holding the solution is put into a water bath with two ice packs, one on each side of the beaker. The concentration of PCL in this solution is 34.0169 mg/ml. The two compositions were electrosprayed as described elsewhere using the following conditions:

| Parameter | Value | |
|---|---|---|
| Pump 1 Flow Rate | 0.5 | ml/hr |
| (Core: PCL Solution) | | |
| Pump 2 Flow Rate | 0.7 | ml/hr |
| (Sheath: Peptide Solution) | | |
| Voltage Applied | 43 | kV |
| Tip to Collector Distance | 160 | mm |

| | |
|---|---|
| Humidity | 30%-44% |

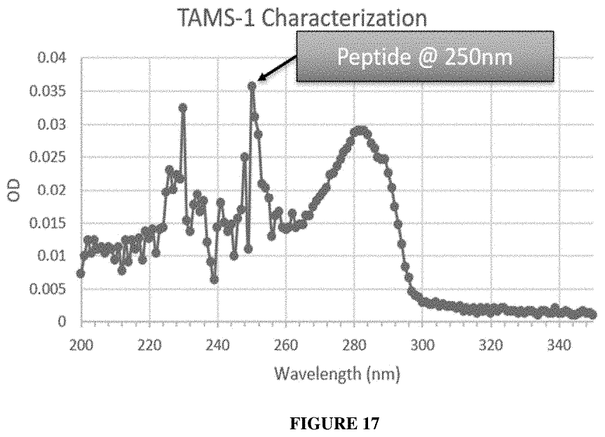

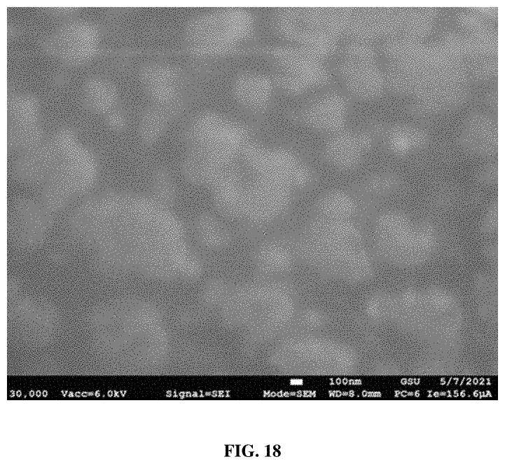

UV-vis spectroscopy indicated the presence of TAMS1 peptides in the nanoparticles (FIG. 17).

The compositions and methods of the appended claims are not limited in scope by the specific compositions and methods described herein, which are intended as illustrations of a few aspects of the claims and any compositions and methods that are functionally equivalent are intended to fall within the scope of the claims. Various modifications of the compositions and methods in addition to those shown and described herein are intended to fall within the scope of the appended claims. Further, while only certain representative compositions and method steps disclosed herein are specifically described, other combinations of the compositions and method steps also are intended to fall within the scope of the appended claims, even if not specifically recited. Thus, a combination of steps, elements, components, or constituents may be explicitly mentioned herein or less, however, other combinations of steps, elements, components, and constituents are included, even though not explicitly stated. The term “comprising” and variations thereof as used herein is used synonymously with the term “including” and variations thereof and are open, non-limiting terms. Although the terms “comprising” and “including” have been used herein to describe various embodiments, the terms “consisting essentially of” and “consisting of” can be used in place of “comprising” and “including” to provide for more specific embodiments of the invention and are also disclosed. Other than in the examples, or where otherwise noted, all numbers expressing quantities of ingredients, reaction conditions, and so forth used in the specification and claims are to be understood at the very least, and not as an attempt to limit the application of the doctrine of equivalents to the scope of the claims, to be construed in light of the number of significant digits and ordinary rounding approaches.

1. A method of forming nanocapsules by pulsed, coaxial electrospraying, the method comprising:
providing at least one core solution comprising at least one solvent and at least one active agent;
providing at least one capsule solution comprising at least one solvent and at least one polymer;
coaxially electrospraying the core solution and capsule solution through a plate that defines a core channel and a capsule channel, the core channel having a core outlet and the capsule channel having a capsule outlet, wherein the capsule outlet is coaxial with the core outlet and is disposed radially outwardly from the core outlet; and
collecting the formed nanocapsules on a grounded collection plate spaced apart from the plate,
wherein the coaxial electrospraying comprises:
applying a pulsating voltage at a frequency to the plate, the pulsating voltage fluctuating between a minimum pulsating voltage and a maximum pulsating voltage, wherein the minimum pulsating voltage is 0 KV or greater and the maximum pulsating voltage is greater than the minimum pulsating voltage; and
applying a constant voltage to the plate, the constant voltage being greater than 0 KV, the maximum pulsating voltage being greater than 0 KV, and a total maximum applied voltage to the plate being the sum of the maximum pulsating voltage and the constant voltage.

2. The method of claim 1, wherein the at least one core solution comprises:
a first core solution comprising at least one solvent and at least one first active agent; and
a second core solution comprising at least one solvent and at least one second active agent,
wherein the method further comprises combining the first core solution and second core solution in a core solution manifold prior to coaxially electrospraying the core solutions through the plate.

3. The method of claim 2, wherein the first core solution is immiscible in the second core solution.

4. The method of claim 2, wherein the first core solution and the second core solution are pumped to the core solution manifold by at least one core solution pump.

5. The method of claim 2, wherein a flow rate of the first core solution into the core solution manifold is greater than a flow rate of the second core solution into the core solution manifold.

6. The method of claim 1, wherein the capsule solution comprises at least:
a first capsule solution comprising at least one solvent and at least one polymer; and
a second capsule solution comprising at least one solvent and at least one additional capsule agent,
wherein the method further comprises combining the first capsule solution and second capsule solution in a capsule solution manifold prior to coaxially electrospraying the capsule solutions through the plate.

7. The method of claim 6, wherein the first capsule solution is immiscible in the second capsule solution.

8. The method of claim 6, wherein the first capsule solution and the second capsule solution are pumped to the capsule solution manifold by at least one capsule solution pump.

9. The method of claim 1, wherein the minimum pulsating voltage is 0 KV and the maximum pulsating voltage is less than or equal to 50 KV.

10. The method of claim 9, wherein the maximum pulsating voltage is less than or equal to 40 KV.

11. The method of claim 1, further comprising sonicating one or more of the core solution, capsule solution, or precursor solutions thereof, prior to co-axial electrospraying.

12. The method of claim 1, wherein the active agent of the core solution comprises at least one chemotherapeutic agent.

13. The method of claim 12, wherein the chemotherapeutic agent comprises paclitaxel.

14. The method of claim 1, wherein the active agent of the core solution comprises a tumor microenvironment (TME) altering agent.

15. The method of claim 14, wherein the TME altering agent comprises GW2580.

16. The method of claim 1, wherein the at least one polymer of the capsule solution comprises one or more biocompatible, biodegradable polymers.

17. The method of claim 16, wherein the biocompatible, biodegradable polymer comprises one or more polyester, mixed polyester, polyanhydride, mixed polyanhydride, poly(ester)anhydride, polysaccharide, polyphosphazene or polyphosphoester.

18. The method of claim 17, wherein the biocompatible, biodegradable polymer comprises PLGA, polycaprolactone, polylactide, polyglycolide, polyhydroxybutyric acid, poly(sebacic acid), poly[1,6-bis(p-carboxyphenoxy)hexane], polyvinylpyrrolidone, polyvinyl alcohol, poly(ethylene oxide), poly(propylene oxide), copolymers thereof, or a mixture thereof.

19-72. (canceled)

73. A nanocapsule, prepared by the process of claim 1.

74. The method of claim 1, wherein the plate comprises a concentric nozzle that defines the core channel and the capsule channel, the concentric nozzle being coupled to and in electrical communication with the plate.

75. (canceled)
